(19) United States
(12) Patent Application Publication
(10) Pub. No.: US 2025/0361527 A1
(43) Pub. Date: Nov. 27, 2025

(54) VECTOR-BASED GENE THERAPY FOR ELONGATOR ACETYLTRANSFERASE COMPLEX SUBUNIT 1 (ELP1)

(71) Applicants: Montana State University, Bozeman, MT (US); University of Massachusetts Chan School of Medicine, Worcester, MA (US)

(72) Inventors: Frances B. Lefcort, Bozeman, MT (US); Claudio Punzo, Worcester, MA (US)

(21) Appl. No.: 19/217,607

(22) Filed: May 23, 2025

Related U.S. Application Data
(60) Provisional application No. 63/651,896, filed on May 24, 2024.

(51) Int. Cl. C12N 15/86; A61K 38/17; A61K 48/00; C07K 14/47

(52) U.S. Cl. CPC C12N 15/86; A61K 38/1709; A61K 48/0058; A61K 48/0075; C07K 14/47; C12N 2750/14122; C12N 2750/14143

(57) ABSTRACT

Viral constructs and methods of their use in the treatment of diseases and disorders mediated by reduced ELP1 expression are provided. The viral constructs include a coding sequence for human ELP1 under control of a heterogenous promoter (such as a small nuclear RNA promoter), allowing for an increase in ELP1 protein expression in neuron cells of the central nervous system, including for instance retinal cells. Methods of using such viral constructs to protect neural cells, for instance by reducing death of retinal ganglion cells, are also provided.

### CROSS-REFERENCE TO RELATED APPLICATION

This application claims priority to and the benefit of the earlier filing of U.S. Provisional Application No. 63/651,896, filed on May 24, 2024, which is incorporated by reference herein in its entirety.

### STATEMENT REGARDING FEDERALLY SPONSORED RESEARCH OR DEVELOPMENT

This invention was made with government support under contract R21 EY031130 awarded by the National Institutes of Health. The government has certain rights in the invention.

### INCORPORATION BY REFERENCE OF SEQUENCE LISTING

A computer readable text file, entitled “M276-6000US.xml” created on or about May 20, 2025, with a file size of 36,864 bytes, contains the sequence listing for this application and is hereby incorporated by reference in its entirety.

### FIELD OF THE DISCLOSURE

The present disclosure relates generally to gene therapy. Specifically, the present disclosure relates to gene therapy to treat diseases or conditions mediated at least in part by under-expression of the ELP1 gene.

### BACKGROUND OF THE DISCLOSURE

Familial dysautonomia (FD) is a rare recessive autonomic and sensory neuropathy. Over 99% of FD patients are homozygous for the “founder” mutation in intron 20 of the Elongator Acetyltransferase Complex Subunit 1 (ELP1) gene (c.2204+6T>C; formerly called “IKBKAP”), causing the “skipping” of exon 20 from the mature mRNA coding sequence (Anderson et al., Am J Hum Genet. 68 (3): 753-758, 2001; Slaugenhaupt et al., Am J Hum Genet, 68 (3): 598-605, 2001). The mutations causing FD are also disclosed in U.S. Pat. No. 7,407,756. This mutant mRNA is then targeted for non-sense mediated decay (Boone et al., PLOS ONE, 5 (12): e15590, 2010). The mis-splicing occurs in a tissue-specific manner, with peripheral neurons being most impacted, producing almost no functional protein (Cuajungco et al., Clinical Autonomic Research, 13 (3): 180-195, 2003; Dietrich et al., Genet Mol Biol, 39 (4): 497-514, 2016). The hallmarks of FD include reduced pain and temperature sensation, gait ataxia, cardiovascular instability, swallowing impairment, gastrointestinal dysfunction, and eventual blindness (Norcliffe-Kaufmann et al., Progress in Neurobiology, 152:131-148, 2017). As patients age, visual impairment becomes one of the most debilitating symptoms, given that it is accompanied by loss of balance and gait ataxia. The optic neuropathy in FD results from the progressive death of retinal ganglion cells (RGCs) and the loss of their axons from the nerve fiber layer (NFL) in the retina's macular region (Kfir et al., Journal of Neurology, 268 (4): 1402-1409, 2021; Mendoza-Santiesteban et al., Journal of Neurology, 261 (4): 702-709, 2014; Mendoza-Santiesteban et al., J Neuropathol Exp Neurol, 76 (3): 238-244, 2017).

Gene therapy is a technique that uses a gene or other expression cassette to treat, prevent, or cure a disease or medical disorder, for instance by viral transduction. Typically, a viral vector has three components: a protein capsid and/or envelope that encapsulates the genetic payload and defines the vector's tissue or cell tropism, as well as its recognition in a biological system; the transgene of interest (the “payload”); and elements that control expression of the transgene. Commonly used viruses for viral transduction include adeno-associated virus (AAV), retroviruses, lentiviruses, and herpes simplex viruses.

It would be beneficial to develop therapeutics to reduce the loss of RGCs that occurs in all FD patients. Being a monogenic disease, FD is a strong candidate for genetic therapies. Gene replacement therapies for retinal diseases show tremendous potential due to the accessibility and immune-privileged status of the eye (Dhurandhar et al., Indian J Ophthalmol, 69 (9): 2257-2265, 2021). Preclinical and clinical studies have demonstrated that adeno-associated viruses (AAVs) are efficient viral vectors for delivering functional genes in hereditary retinal diseases (Dhurandhar et al., Indian J Ophthalmol, 69 (9): 2257-2265, 2021; Bennett & Maguire, Cold Spring Harb Perspect Med, 2022; Li & Samulski, et al., Nature Reviews Genetics, 21 (4): 255-272, 2020). However, to date, no gene therapy approaches have been used to introduce wild-type copies of ELP1 to correct disease phenotypes.

### SUMMARY OF THE DISCLOSURE

Described herein are data showing the effectiveness of human ELP1 gene therapy in preventing RGC death in FD mouse models.

Isolated nucleic acid molecules are described, which include a polynucleotide including a nucleic acid sequence encoding a human ELP1 (hELP1) protein; a promoter sequence arranged to promote expression of the human ELP1 protein in a mammalian cell; and a post-transcriptional regulatory element. In example of the isolated nucleic acid molecule, the promoter sequence promotes expression of the ELP1 protein in a mammalian neuron. For instance, the promoter sequence promotes expression of the ELP1 protein in a central nervous system (CNS) neuron, such as in a retinal neuron.

Also provided are recombinant AAV (rAAV) virions that include: an AAV2 capsid; and a nucleic acid molecule of the described nucleic acid embodiments.

Another embodiment is a recombinant AAV (rAAV) virion which includes an AAV2 capsid; and a recombinant polynucleotide including a nucleic acid sequence encoding human ELP1 operably linked to a promoter that promotes expression of the human ELP1 in retinal neurons, flanked by AAV ITR sequences.

Also provided are pharmaceutical compositions that include at least one rAAV of any of the described embodiments, formulated for administration to a mammalian subject. By way of example, such pharmaceutical compositions are in some cases formulated for ocular administration. Others are formulated for systemic or local administration outside of the eye.

Yet another embodiment is use of at least one rAAV of any of the provided rAAV embodiments, or a composition of any of the composition embodiments, to express hELP1 protein in a cell the genome of which contains a mutated hELP1 gene. By way of example, in such uses the cell is a central nervous system cell or a peripheral nervous system cell of a mammal. In specific examples, the cell is a neuron, such as a retinal neuron.

Also provided are methods of treating ELP1 deficiency in a mammalian subject, the methods including: administering to the mammalian subject a pharmaceutical formulation including an AAV2-hELP1 vector including: a AAV2 capsid, and a recombinant polynucleotide including: a nucleic acid sequence encoding human ELP1 operably linked to a promoter that promotes expression of the human ELP1 in retinal neurons, flanked by AAV ITR sequences; wherein the AAV2-hELP1 vector is delivered to at least one target site in the subject.

Another embodiment is a method of treating ELP1 deficiency in a mammalian subject, which method includes providing a pharmaceutical formulation including an AAV2-hELP1 vector including: a AAV2 capsid, and a recombinant DNA insert including a nucleic acid sequence encoding human ELP1; and delivering the pharmaceutical formulation to at least one target site in the central nervous system (CNS) or in the peripheral nervous system (PNS) of the subject.

Also provided are methods of increasing ELP1 protein level in a central nervous system (CNS) cell or a peripheral nervous system (PNS) cell of a mammal, which methods include administering to the CNS or the PNS of the mammal a viral expression vector (such as an AAV2 viral expression vector) including: a human ELP1-encoding nucleic acid sequence; and a small nuclear RNA U1a promoter arranged to promote expression of the human ELP1 protein in a cell of the CNS or the PNS.

Also provided are method of increasing ELP1 protein level in a central nervous system (CNS) cell or a peripheral nervous system (PNS) cell of a mammal, essentially as described herein. Yet another embodiment is a recombinant AAV (rAAV) virion, or an ELP1-expressing rAAV vector, essentially as described herein.

### BRIEF DESCRIPTION OF THE DRAWINGS

FIG. 1A: Experimental workflow. Mice were intravitreally injected with an AAV2-vector in both eyes at P21, and the retinal ganglion cell (RGC) number was quantified 3 months later. An experimental or control virus was injected into the vitreous humor of the mouse eye, where it can diffuse into the retina and target RGCs. FIG. 1B shows a schematic representation of a AAV2-vector, such as that used in Example 1.

FIGS. 2A-2B: Viral transduction of murine Elp1 rescues RGCs in a mouse model of FD. (FIG. 2A) Representative retinal whole mount (top) and peripheral retina (bottom) 3 months after intravitreal injection with AAV2-U1a-mElp1. RGCs were immunolabeled using an antibody against Brn3a. Scale bar, 500 μm. (FIG. 2B) Quantification of Brn3a+RGCs from control mice (dark gray) compared to FD mice (lighter gray gradient) receiving injections at P21 with either the eGFP (1.6×10⁹ vg/eye) or experimental mElp1 virus (1.6×10⁹ vg/eye, 1.7×10⁹ vg/eye, 8.4×10⁸ vg/eye). Brn3a+ cells were counted in a 1 mm² area in each quadrant of the peripheral retina. The adjusted p-values are displayed. ns p=0.2, 0.99, *p=0.02, 0.04, ****p<0.0001, one-way ANOVA with Tukey's multiple comparisons follow-up test. Data are shown as average±SEM, and each data point represents an individual retina. The sample size for individual groups is represented by n within each bar.

FIGS. 3A-3C: In vivo expression of human ELP1 prevents loss of RGCs in a mouse model of FD. (FIG. 3A) Representative retinal whole mount (top) and peripheral retina (bottom) 3 months after intravitreal injections with hELP1. RGCs were immunolabeled using an antibody against Brn3a. Scale bar, 250 μm. (FIG. 3B) Quantification of Brn3a+RGCs from control littermate mice (dark gray) compared to FD mice (lighter gray gradient) receiving injections at P21 with either the eGFP (8.0×10⁸ vg/eye) or experimental ELP1 (5.4×10⁸ vg/eye, 2.7×10⁸ vg/eye, 2.7×10⁷ vg/eye) virus. Brn3a+cells were counted in a 1 mm² area in each quadrant of the peripheral retina. (FIG. 3C) Representative images of Brn3a+ RGCs (gray, asterisks represent lower Brn3a intensities) and the human ELP1 protein expression (arrows) 3 months after intravitreal injections into mutant mice. Scale bar, 25 μm. The adjusted p-value is displayed. *p=0.02, ns=0.818, ns=0.057; unpaired, two-sided Mann-Whitney U test. There is a statistically significant difference between the untreated FD mice and FD mice receiving the higher dose of AAV2-hELP1 5.4×10⁸ vg/eye. Data are shown as average ±SEM, and each data point represents an individual retina. The sample size for individual groups is represented by n within each bar.

FIGS. 4A-4C: (FIG. 4A) Representative immunoblot showing the expression of murine Elp1 in two different cell lines 4 days post-transduction with AAV2-U1a-mElp1 and AAV2-U1a-eGFP. This is a compiled image from two discontinuous gels. There is an increase in Elp1 expression in the transduced cells. 20 μg of protein was loaded in each well, and GAPDH was used as the loading control. (FIG. 4B) Densitometric analysis of Elp1 protein in HEK293 and CHO cells transduced with AAV2-U1a-mElp1 compared to untreated cells (HEK293: **p=0.004, 0.005; CHO: **p=0.001, ***p=0.0008, two-way ANOVA with Tukey's multiple comparisons follow-up test. The experiment was done in triplicate. (FIG. 4C) RT-qPCR analysis of Elp1 expression in retinal homogenates 1 month after intravitreal injections of AAV2-U1a-mElp1. Only Elp1 is strongly increased.

FIGS. 5A-5C: (FIG. 5A) Representative immunoblot showing the upregulation of human ELP1 in HEK293 cells 4 days post-transduction with AAV2-U1a-hELP1. Note the gradual increase in ELP1 expression as the multiplicity of infection (m.o.i.) increases. Equal amounts of protein were loaded in each well, and GAPDH was used as the loading control. This is a cropped gel image. (FIG. 5B) Densitometric analysis of ELP1 protein in HEK293 cells transduced with AAV2-U1a-hELP1 compared to untreated cells. The experiment was done five times. (FIG. 5C) RT-qPCR analysis of ELP1 expression in retinal homogenates (n=2) 1 month after intravitreal injections of AAV2-U1a-hELP1. As expected, no expression of the human ELP1 gene was seen in the uninjected control homogenates, whereas the control mice receiving AAV2-U1a-hELP1 showed increasing levels of human ELP1 protein or mRNA.

FIGS. 6A-6C: Representative immunoblot showing full gel images. (FIG. 6A) The full gel image from FIG. 4A uses a 7.5% gel showing the expression of murine Elp1 in HEK293 and CHO cells. (FIG. 6B) The full gel image from FIG. 4B uses a 12% gel showing the expression of GFP in HEK293 and CHO cells. (FIG. 6C) The full gel image from FIG. 5A shows GFP expression in HEK293 cells.

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. In the Sequence Listing:

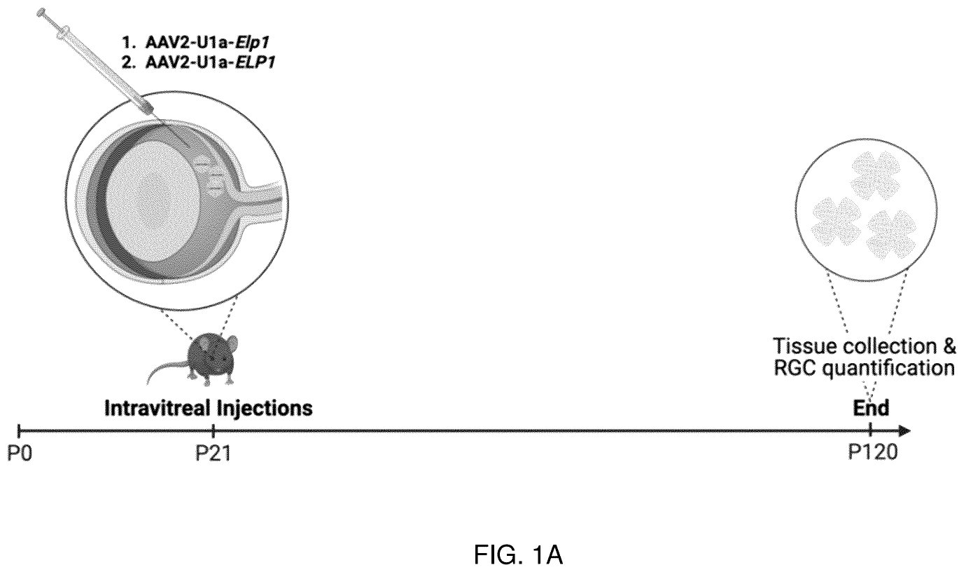

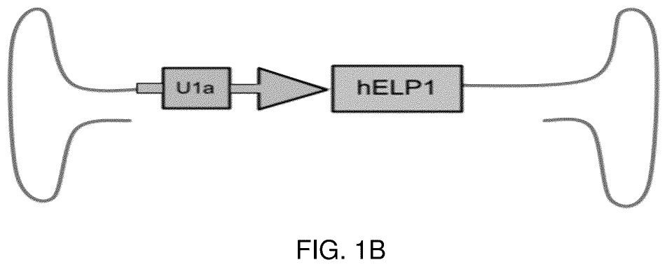

SEQ ID NO: 1 is the full recombinant AAV2-U1a-hELP1 nucleotide sequence; this construct is illustrated in the schematic of FIG. 1B.

SEQ ID NO: 2 is an inverted terminal repeat (ITR) BP (at positions 7-147 of SEQ ID NO: 1), in the 5′ strand of AAV2-U1a-hELP1.

SEQ ID NO: 3 is an inverted terminal repeat (ITR) BP (at positions 2745-2885 of SEQ ID NO: 1) 5′ strand of AAV2-U1a-hELP1.

SEQ ID NO: 4 Murine promoter Ula (at positions 2898-3148 of SEQ ID NO: 1), 5′ strand of AAV2-U1a-hELP1.

SEQ ID NO: 5 is the nucleotide sequence of Human Ula cDNA (GenBank NM_004596). In this sequence, the CDS of Homo sapiens small nuclear ribonucleoprotein polypeptide A (SNRPA) is at positions 207-1055. Presumptively, promoter element(s) are present within the first 206 nucleotides. See, for instance, Nelissen et al., Gene 102 (3): 189-196, 1991.

SEQ ID NO: 6 is the nucleotide sequence of a human ELP1 cDNA (at positions 3161-7156 of SEQ ID NO: 1), 5′ strand of AAV2-U1a-hELP1.

SEQ ID NO: 7 is the amino acid sequence of human ELP1 protein (encoded by SEQ ID NO: 6).

SEQ ID NO: 8 is the nucleotide sequence of sPA (at positions 7166-7213 of SEQ ID NO: 1) 5′ strand of AAV2-U1a-hELP1.

SEQ ID NOs: 9-24 are primer sequences, in accord with the following information:

| Sequence Name | | Seq 5′ to 3′ | Accession No. | SEQ ID NO: |
|---|---|---|---|---|
| ELP1 | F | CTCTGCAGTCTCAGCACACA | NM_003640.5 | SEQ ID NO: 9 |
| ELP1 | R | CTGCTCCAGGATTGGCTCAA | | SEQ ID NO: 10 |
| Elp1 | F | GGTGACAGTCTTTCGGCAGA | NM_026079.3 | SEQ ID NO: 11 |
| Elp1 | R | GATCAGCAGCCGGTAGGTAC | | SEQ ID NO: 12 |
| Cckar | F | TGAACAAACGCTTTCGCCTG | NM_009827 | SEQ ID NO: 13 |
| Cckar | R | TGGCTGTAGGAATACCGGGA | | SEQ ID NO: 14 |
| Ptger4 | F | CACCACCTCGCTGAGAACTT | NM_008965 | SEQ ID NO: 15 |
| Ptger4 | R | TCCTTTAGAGGCAGGCTCCT | | SEQ ID NO: 16 |
| Hprt | F | TCAGTCAACGGGGGACATAAA | NM_013556 | SEQ ID NO: 17 |
| Hprt | R | GGGGCTGTACTGCTTAACCAG | | SEQ ID NO: 18 |
| Actb | F | AACCCTAAGGCCAACCGTGAA | NM_007393 | SEQ ID NO: 19 |
| Actb | R | TCACGCACGATTTCCCTCTCA | | SEQ ID NO: 20 |
| Bdnf | F | ACTGCAGTGGACATGTCTGG | NM_007540 | SEQ ID NO: 21 |
| Bdnf | R | AGTTGGCCTTTGGATACCGG | | SEQ ID NO: 22 |
| hELP1 | F | CCTGAGCAGCAATCATGTG | NM_003640 | SEQ ID NO: 23 |
| hELP1 | R | TACATGGTCTTCGTGACATC | | SEQ ID NO: 24 |

### DETAILED DESCRIPTION

Provided herein is an isolated nucleic acid molecule including: a polynucleotide including a nucleic acid sequence encoding a human ELP1 (hELP1) protein; a promoter sequence arranged to promote expression of the human ELP1 protein in a mammalian cell; and a post-transcriptional regulatory element. In example of the isolated nucleic acid molecule, the promoter sequence promotes expression of the ELP1 protein in a mammalian neuron. For instance, the promoter sequence promotes expression of the ELP1 protein in a central nervous system (CNS) neuron, such as in a retinal neuron.

In examples of the provided isolated nucleic acid molecule, the isolated nucleic acid molecule includes a recombinant AAV2 vector, and the polynucleotide is flanked by AAV ITR sequences.

Also described are embodiments of the isolated nucleic acid molecule, wherein the encoded human ELP1 protein includes an amino acid sequence at least 95% identical to the sequence of SEQ ID NO: 7; and/or wherein the nucleotide sequence encoding the human ELP1 protein has a sequence at least 95% identical to the sequence of SEQ ID NO: 6; and/or wherein the nucleotide sequence encoding the human ELP1 protein includes the sequence of SEQ ID NO: 6.

By way of example in any of the provided isolated nucleic acid molecule embodiments, the promoter sequence may be no more than about 300 nucleotides long. For instance, the promoter sequence is from a small nuclear RNA protein (SNRPA), such as a small nuclear RNA U1 promoter, in exemplary embodiments. In specific instances, the promoter sequence is a murine or human Ula promoter. Described example promoters include a promoter having a sequence including or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell.

Examples of the described isolated nucleic acid molecule embodiments include instances wherein the polynucleotide molecule includes: a nucleotide sequence at least 95% identical to SEQ ID NO: 6 and encoding a functional human ELP1 protein; and a Ula promoter sequence arranged to promote expression of the human ELP1 protein. In additional examples, the polynucleotide molecule includes: a nucleotide sequence identical to SEQ ID NO: 6; a U1a promoter sequence having the sequence SEQ ID NO: 4 arranged to promote expression of the human ELP1 protein; or both.

In representative isolated nucleic acid molecule embodiments, the polynucleotide molecule has a sequence at least 95% identical to SEQ ID NO: 1, or alternatively the polynucleotide molecule has a sequence identical to SEQ ID NO: 1.

Also provided are recombinant AAV (rAAV) virions that include: an AAV2 capsid; and a nucleic acid molecule of the described nucleic acid embodiments.

Another embodiment is a recombinant AAV (rAAV) virion which includes an AAV2 capsid; and a recombinant polynucleotide including a nucleic acid sequence encoding human ELP1 operably linked to a promoter that promotes expression of the human ELP1 in retinal neurons, flanked by AAV ITR sequences.

Yet another embodiment is a recombinant adeno-associated serotype 2 (rAAV2) virion that includes a rAAV2 vector polynucleotide; and, contained therein: a human ELP1 gene nucleic acid sequence (hELP1); a Ula promoter sequence arranged to promote expression of the human ELP1 protein; and a post-transcriptional regulatory element.

In any of the provided rAAV embodiments, optionally the polynucleotide includes a nucleotide sequence functionally equivalent to the nucleotide sequence of SEQ ID NO: 1 (rAAV2.U1a.hELP1).

Also provided are pharmaceutical compositions that include at least one rAAV of any of the described embodiments, formulated for administration to a mammalian subject. By way of example, such pharmaceutical compositions are in some cases formulated for ocular administration. Others are formulated for systemic or local administration outside of the eye.

Yet another provided embodiment is use of at least one rAAV of any of the provided rAAV embodiments, or a composition of any of the composition embodiments, to express hELP1 protein in a cell the genome of which contains a mutated hELP1 gene. By way of example, in such uses the cell is a central nervous system cell or a peripheral nervous system cell of a mammal. IN specific examples, the cell is a neuron, such as a retinal neuron.

Also provided are methods of treating ELP1 deficiency in a mammalian subject, the methods including: administering to the mammalian subject a pharmaceutical formulation including an AAV2-hELP1 vector including: a AAV2 capsid, and a recombinant polynucleotide including: a nucleic acid sequence encoding human ELP1 operably linked to a promoter that promotes expression of the human ELP1 in retinal neurons, flanked by AAV ITR sequences; wherein the AAV2-hELP1 vector is delivered to at least one target site in the subject.

Yet another embodiment is a method of increasing ELP1 expression in retinal cells of a mammalian subject in need thereof, the method including: administering to the mammalian subject a pharmaceutical formulation including an AAV2-hELP1 vector including: a AAV2 capsid, and a recombinant polynucleotide including: a nucleic acid sequence encoding human ELP1 operably linked to a promoter that promotes expression of the human ELP1 in retinal neurons, flanked by AAV ITR sequences; wherein the AAV2-hELP1 vector is delivered to at least one target site in the subject. In examples of this embodiment, one or more retinal cells of the mammalian subject express a mutant ELP1 protein; one or more retinal cells of the mammalian subject exhibit reduced expression of ELP1 as compared to a normal subject; and/or one or more retinal cells of the mammalian subject express an ELP1 protein with reduced functionality as compared to a normal subject. By way of example within this embodiment, administering the viral expression vector results in some instances in enhanced survival and/or functionality of retinal cells of the mammalian subject.

Another embodiment is a method of treating ELP1 deficiency in a mammalian subject, which method includes: providing a pharmaceutical formulation including an AAV2-HELP1 vector including: a AAV2 capsid, and a recombinant DNA insert including a nucleic acid sequence encoding human ELP1; and delivering the pharmaceutical formulation to at least one target site in the central nervous system (CNS) or in the peripheral nervous system (PNS) of the subject.

Yet another embodiment is a method of increasing ELP1 expression in cells of a mammalian subject in need thereof, the method including: providing a pharmaceutical formulation including an AAV2-hELP1 vector including: a AAV2 capsid, and a recombinant DNA insert including a nucleic acid sequence encoding human ELP1; and delivering the pharmaceutical formulation to at least one target site in the central nervous system (CNS) or in the peripheral nervous system (PNS) of the subject. In examples of this embodiment, one or more retinal cells of the mammalian subject express a mutant ELP1 protein; one or more retinal cells of the mammalian subject exhibit reduced expression of ELP1 as compared to a normal subject; and/or one or more retinal cells of the mammalian subject express an ELP1 protein with reduced functionality as compared to a normal subject. By way of example within this embodiment, administering the viral expression vector results in some instances in enhanced survival and/or functionality of retinal cells of the mammalian subject.

In any of the provide methods embodiments, the target site may include at least one eye of the subject. In such instances, the pharmaceutical formulation can be administered to the eye of the mammalian subject by an intravitreal, suprachoroidal, subretinal, or intraocular route, optionally by injection.

In any of the provide methods embodiments, the method can include delivering to the subject a dose of 1×10⁹-1×10¹² viral genomes (vg)/eye; 1×10¹⁰-1×10¹¹ vg/eye; or 1×10⁹ 1×10¹⁰ vg/eye.

Further, in examples of the provide methods embodiments, treating ELP1 deficiency in the mammalian subject reduces or prevents death of a neuron in the CNS or in the PNS of the mammalian subject.

In any of the provide methods embodiments, it is contemplated that the mammalian subject can be a human subject. Optionally, the human subject is diagnosed with familial dysautonomia (FD), is suspected of carrying a mutation in their ELP1 gene, and/or has a mutated ELP1 gene.

In examples of the provide methods embodiments, the nucleic acid sequence encoding hELP1 is an hELP1 cDNA; and/or the recombinant DNA insert includes from 5′ to 3′: a first inverted terminal repeat (ITR), a small nuclear RNA U1a promoter, the nucleic acid sequence encoding functional human ELP1, a poly A signal sequence, and a second ITR; and/or the recombinant DNA insert includes: a Ula promoter including or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell, and a nucleic acid sequence at least 95% identical to SEQ ID NO: 6; and/or the AAV2-hELP1 vector includes the nucleotide sequence of SEQ ID NO: 1.

Further, in provided method embodiments, optionally the pharmaceutical formulation is delivered by injection or infusion; and/or the pharmaceutical formulation is delivered to eye of the subject by an intravitreal, suprachoroidal, subretinal, or intraocular route, optionally by injection.

Also provided are methods of increasing ELP1 protein level in a central nervous system (CNS) cell or a peripheral nervous system (PNS) cell of a mammal, which methods include administering to the CNS or the PNS of the mammal a viral expression vector (such as an AAV2 viral expression vector) including: a human ELP1-encoding nucleic acid sequence; and a small nuclear RNA Ula promoter arranged to promote expression of the human ELP1 protein in a cell of the CNS or the PNS. In examples of such method embodiment, the human ELP1-encoding nucleic acid has a sequence at least 95% identical to the sequence of SEQ ID NO: 6. Optionally, the human ELP1-encoding nucleic includes the sequence of SEQ ID NO: 6.

Yet another embodiment is a method of increasing ELP1 protein expression in a central nervous system (CNS) cell or a peripheral nervous system (PNS) cell of a mammalian subject, including administering to the CNS or the PNS of the mammalian subject a viral expression vector including: a human ELP1-encoding nucleic acid sequence; and a small nuclear RNA U1a promoter arranged to promote expression of the human ELP1 protein in a cell of the CNS or the PNS. In examples of this embodiment, one or more CNS cells or PNS cells of a mammalian subject express a mutant ELP1 protein; one or more CNS cells or PNS cells of a mammalian subject exhibit reduced expression of ELP1 as compared to a normal subject; and/or one or more CNS cells or PNS cells of the mammalian subject express an ELP1 protein with reduced functionality as compared to a normal subject. By way of example within this embodiment, administering the viral expression vector results in some instances in enhanced survival and/or functionality of retinal cells of the mammalian subject.

Also, in examples of the methods of increasing ELP1 protein level, the encoded human ELP1 protein has a sequence at least 95% identical to the sequence of SEQ ID NO: 7. Optionally, the encoded human ELP1 protein includes the sequence of SEQ ID NO: 7.

In examples of the methods of increasing ELP1 protein level, the promoter is a ubiquitous promoter or a retinal cell-specific promoter. For instance, in examples the promoter is a murine Ula promoter or a human Ula promoter; and/or the promoter has a sequence including or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell.

In any of the provided methods of increasing ELP1 protein level examples, optionally the viral vector includes an adeno-associated virus serotype 2 (AAV2) or an adeno-associated virus serotype 9 (AAV9) vector. For instance, in some cases the viral vector is an AAV2 vector, and the AAV2 vector includes AAV2-U1a-Elp1.

Also provided are method of increasing ELP1 protein level in a central nervous system (CNS) cell or a peripheral nervous system (PNS) cell of a mammal, essentially as described herein.

Yet another embodiment is a recombinant AAV (rAAV) virion, or an ELP1-expressing rAAV vector, essentially as described herein.

More generally, constructs include a viral particle (including recombinant AAV2 viral particles), and viral nucleic acid constructs, including recombinant viral genomes that comprise, a promoter, a transgene, and optionally a post-transcriptional regulatory sequence. Promoters may be ubiquitous or cell/tissue specific (e.g., neuronal or retinal specific promoters). Exemplary viral vectors include adeno-associated virus (AAV), such as AAV serotype 2 (AAV2) or AAV serotype 9 (AAV9) virus vectors. Exemplary promoters include ubiquitous and cell (or tissue) specific promoters.

The viral particles may be administered concurrently or sequentially with a therapeutic agent. For example, for concurrent administration, the recombinant AAV may be formulated with a therapeutic agent in a single composition suitable for delivery, for example, injection, by methods known in the art. Alternatively, the AAV may be injected (or otherwise delivered) in separate compositions, simultaneously or sequentially with another therapeutic agent.

Embodiment of formulations for administration include a pharmaceutically and/or physiologically acceptable vehicle, diluent, carrier, or excipient, such as buffered saline or other buffers, e.g., HEPES, to maintain physiologic pH. For a discussion of such components and their formulation, see, generally, Gennaro, A E., Remington: The Science and Practice of Pharmacy, Lippincott Williams & Wilkins Publishers; 2003 or latest edition). See also, WO 2000/015822A1. If the preparation is to be stored for long periods, it may be frozen, for example, in the presence of glycerol.

Examples of nucleic acids suitable for use in the methods described herein include, but are not limited to, viral vectors (such as recombinant AAV genomes) encoding a human ELP1. Particularly viral vectors are rAAV2 vectors (e.g., recombinant viral genomes with AAV2 ITR sequences) that encode hELP1 for expression in a neuron, such as a CNS neuron, and particularly neurons in the retina of a mammal. In embodiments, such expression reduces ganglion cell death.

Aspects of the current disclosure are now described with additional details and options as follows: (I) Vectors; (II) Familial Dysautonomia; (III) Pharmaceutical Compositions; (IV) Kits; (V) Representative Definitions; (VI) Exemplary Embodiments; (VII) Experimental Examples; and (VIII) Closing Paragraphs. These headings do not limit the interpretation of the disclosure and are provided for organizational purposes only.

### (I) VECTORS

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell. Useful vectors include, for example, plasmids (e.g., DNA plasmids or RNA plasmids), transposons, cosmids, bacterial artificial chromosomes, and viral vectors, such as the recombinant AAV particles described herein.

The term “nucleic acid cassette” or “expression cassette” as used herein refers to genetic sequences within the vector that can express RNA, and subsequently a polypeptide. In one aspect, the nucleic acid cassette contains one or more gene(s)-of-interest, e.g., a polynucleotide(s)-of-interest. In another aspect, the nucleic acid cassette contains one or more expression control sequences and one or more gene(s)-of-interest. Vectors may include one, two, three, four, five, or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred aspect of the disclosure, the nucleic acid cassette contains the sequence of a plurality of therapeutic genes used to treat, prevent, or ameliorate a genetic disorder, such as an ocular disorder. The cassette can be removed and inserted into a plasmid or viral vector as a single unit.

As will be evident to one of skill in the art, the term “viral vector” is widely used to refer either to a nucleic acid molecule (e.g., a transfer plasmid) that includes virus-derived nucleic acid elements that typically facilitate the transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “adeno-associated viral vector” (AAV) refers to a viral vector or plasmid containing structural and functional genetic elements, or portions thereof, that are primarily derived from an adeno-associated virus.

In particular aspects, the terms “viral vector” and “viral expression vector” may be used to refer to viral transfer plasmids and/or infectious viral particles. Where reference is made herein to elements such as cloning sites, promoters, regulatory elements, heterologous nucleic acids, etc., it is to be understood that the sequences of these elements are present in RNA form in the viral particles of the disclosure and are present in DNA form in the DNA plasmids of the disclosure. Nucleic acid expression vectors suitable for use in gene therapy are known in the art. For example, the nucleic acid expression vector is a viral vector.

Adenovirus vectors and adeno-associated virus vectors may be used with the compositions and methods of the disclosure. The adenovirus vector may result in a shorter-term expression (e.g., less than about a month) than adeno-associated virus, in some aspects, and may exhibit much longer expression. The adeno-associated virus (AAV) payload capacity is about 5 kb. Thus, the sequence of transgene, and the size and type of regulatory elements may be varied depending on the viral vector being used. In some aspects, the viral vector may be selected based on its ability to transduce (a.k.a., infect) a particular cell type.

AAV is a naturally replication-deficient virus that requires a helper virus for replication. This self-limiting infection coupled with the ability to stably infect dividing and non-dividing cells makes AAV a useful target for in vivo gene therapy. Further, AAV vectors have tissue-targeting abilities that can be enhanced with capsid re-engineering. For example, D100 is a recently developed AAV vector (Kotterman et al., bioRxiv 2021.06.24.449775 doi.org/10.1101/2021.06.24.449775 (2021)). In some aspects, the vector is recombinant AAV-2 (rAAV2). See also U.S. Pat. Nos. 11,197,937 and 10,982,228.

### Promoters

The term “promoter” as used herein refers to a recognition site of a polynucleotide (DNA or RNA) to which an RNA polymerase binds. The term “enhancer” refers to a segment of DNA that contains sequences capable of providing enhanced transcription. In some instances, the enhancement can function independently of its orientation relative to another control sequence. An enhancer can function cooperatively or additively with promoters and/or other enhancer elements. The term “promoter/enhancer” refers to a segment of DNA that contains sequences capable of providing both promoter and enhancer functions.

In some embodiments, the expression of the therapeutic transgene is driven by a ubiquitous promoter, i.e., cytomegalovirus immediate enhancer/B-actin (CAG), cytomegalovirus (CMV), synapsin, elongation factor-1 alpha (EF1a), or Thymocyte differentiation antigen 1 (Thy-1) promoter or long terminal repeat (LTR). In specific embodiments, the promoter is a small nuclear ribonucleoprotein polypeptide A (SNRPA) promoter from a mammal, such as murine or human SNRPA promoters. By way of example, the promoter is a murine or human Ula promoter such as those illustrated herein.

In other embodiments, the promoter is an inducible or a cell-specific promoter. Cell type-specific promoters that enable transgene expression in specific subpopulations of cells, i.e., retinal neuron cells or degenerating cells, may be preferred. These cells may include, but are not limited to, a retinal ganglion cell. Cell type-specific promoters are well known in the art. Cell type-specific promoters may be modified using recombinant DNA techniques known in the art to increase efficiency of expression and selective targeting.

The term “operably linked”, refers to a juxtaposition wherein the components described are in a relationship permitting them to function in their intended manner. In one aspect, the term refers to a functional linkage between a nucleic acid expression control sequence (such as a promoter, and/or enhancer or other expression control sequence) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively. Illustrative ubiquitous expression control sequences include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, synapsin promoter, Thy-1 promoter, CaMKlla promoter, NK-3, and Pcp2 (L7), SNCG, HKamac, and Rho and a β-actin promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain aspects of the disclosure provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Generally, promoter sequences and/or any associated regulatory sequences may include about at least 150 bp, 200 bp, 300 bp, 400 bp, 500 bp, 600 bp, 700 bp, 800 bp, 900 bp, 1000 bp, 2000 bp, 3000 bp, 4000 bp, 5000 bp or 10000 bp. Promoter sequences and any associated regulatory sequences may include about at most 150 bp, 200 bp, 300 bp, 400 bp, 500 bp, 600 bp, 700 bp, 800 bp, 900 bp, 1000 bp, 2000 bp, 3000 bp, 4000 bp, 5000 bp or 10000 bp.

### Transgenes

The transgene of interest (i.e., therapeutic transgene) can be any Elongator Acetyltransferase Complex Subunit 1 (ELP1) encoding sequence, though a human ELP1 (hELP1) encoding sequence is preferred. Example human ELP1 protein and encoding nucleic acid sequences are provided herein. See also U.S. Pat. No. 7,407,756.

In some embodiments, a polyA signal sequence may be inserted downstream of the transgene in an expression cassette or nucleic acid expression vector of the present disclosure. Including a polyA signal sequence can be beneficial as it avoids the need to include a polyA tail as part of the transgene sequence.

Alternatively, the transgene can include a polyA tail. Suitable polyA tails are known in the art, and include, for example, human growth hormone poly A tail (hGHpA), bovine growth hormone polyA tail (bGHpA), bovine polyA, SV40 polyA, and AV40 pA. A polyA sequence may include a length of 1-10 bp, 10-20 bp, 20-50 bp, 50-100 bp, 100-500 bp, or 500 bp-1 kb in length. A polyA sequence may include a length of at least 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, or 1 kb in length. A polyA sequence may include a length of at most 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, or 1 kb in length. The length of polyA sequence included may be influenced by the load capacity of the viral vector selected.

In some cases, polyA sequences may be optimized for various parameters affecting protein expression, including the mRNA half-life of the transgene in the cell, stability of the mRNA of the transgene, or transcriptional regulation. For example, polyA sequences may be altered to increase mRNA transcript of the transgene, which may result in increased protein expression. In some cases, the polyA sequences may be altered to decrease the half-life of the mRNA transcript of the transgene, which may result in decreased protein expression.

### Post-Transcriptional Regulatory Elements

In particular aspects, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE) (Zufferey et al., J. Virol., 73:2886, 1999); (Huang et al., Mol. Cell. Biol., 15 (7): 3864, 1995; and the like (Liu et al., Genes Dev., 9:1766, 1995), or modified WPRE.

Elements directing the efficient termination and polyadenylation of a heterologous nucleic acid transcript increase heterologous gene expression. Transcription termination signals are generally found downstream of the polyadenylation signal. The terms “polyA tail”, “polyA site” or “polyA signal sequence” as used herein denotes a DNA sequence that directs both the termination and polyadenylation of the nascent RNA transcript by RNA polymerase II. Efficient polyadenylation of the recombinant transcript is desirable as transcripts lacking a poly A tail are unstable and are rapidly degraded. Illustrative examples of polyA signals that can be used in a vector include an ideal polyA sequence, a human growth hormone polyadenylation signal (hGHpA), bovine growth hormone polyA sequence (BGHpA), a rabbit β-globin polyA sequence (rβgpA), or mouse growth hormone polyA sequence (mGHpA) or another suitable heterologous or endogenous polyA sequence known in the art.

In certain aspects, the viral vector further includes one or more insulator elements. Insulator elements may contribute to protecting lentivirus-expressed sequences, e.g., therapeutic polypeptides, from integration site effects, which may be mediated by cis-acting elements present in genomic DNA and lead to deregulated expression of transferred sequences (i.e., position effect; see, e.g., Burgess-Beusse et al., Proc. Natl. Acad. Sci., USA, 99:16433, 2002; and Zhan et al., Hum. Genet., 109:471, 2001). In some aspects, transfer vectors include one or more insulator elements the 3′ LTR, and upon integration of the provirus into the host genome, the provirus includes the one or more insulators at both the 5′ LTR or 3′ LTR, by virtue of duplicating the 3′ LTR. Suitable insulators for use in the disclosure include the chicken β-globin insulator (see, e.g., Chung et al., Cell 74:505, 1993; Chung et al., PNAS 94:575, 1997; and Bell et al., Cell 98:387, 1999). Examples of insulator elements include an insulator from an Bglobin locus, such as chicken HS4.

### (III) FAMILIAL DYSAUTONOMIA

Familial dysautonomia (FD), also known as Riley Day syndrome or hereditary sensory and autonomic neuropathy III (HSAN-III), is the best known and most common member of a group of congenital sensory and autonomic neuropathies (HSAN) characterized by widespread sensory and variable autonomic dysfunction. FD affects neuronal development and is associated with progressive neuronal degeneration. Multiple systems are impacted resulting in a markedly reduced quality of life and premature death. FD is caused by mutations in the IKBKAP gene and all cases described to date involve an intron 20 mutation that results in a unique pattern of tissue-specific exon skipping. See also, for example, Shetty et al., Human Molecular Genetics, 2011, 20 (21): 4093-4101, 2011; Axelrod et al., PediatricResearch, 70 (5): 480-483, 2011; Gold-von Simson et al., PediatricResearch, 65 (3): 341-346, 2009; Yoshida et al., PNAS, 112 (9): 2764-2769, 2015; and International Patent Publications WO 2015/005491, WO 2010/118367, and WO 2014/124458; and U.S. Pat. No. 7,407,756.

The viral-mediated hELP1 gene therapy of the present disclosure may be useful for the treatment, prevention, and amelioration of one or more symptoms or conditions associated with FD. Though exemplified herein through treatment of the retina, it is also believed that the provided hELP1 gene therapy will be beneficial systemically, for instance when administered (e.g., injected) to target the peripheral nervous system (e.g., by local injection (such as spinal injection), intravenous injection, intrathecally, and so forth). Thus, embodiments of the current disclosure are directed to treating or prevent optical or other central nervous system (CNS) aspects of FD; while other embodiments focus on treating peripheral nervous system aspects of FD. In each instance, the transgene encodes a human ELP1 protein, thereby providing a higher level of ELP1 protein to target cells in either the CNS or the PNS (or both, in some cases).

Methods of diagnosing FD are known to those in the field and include analysis of characteristic signs and symptoms in the subject. Clinical diagnosis may rely on a constellation of criteria, including: no fungiform papillae on the tongue, decreased deep-tendon reflexes, lack of an axon flare following intradermal histamine, and no overflow tears with emotional crying. In addition, genetic testing can be carried out to detect the underlying causative IKBKAP gene mutation (Blumenfeld et al., Nature Genetics, 4:160-165, 1993; News Roundup, BMJ 322 (7282): 319, 2001). Other methods are also available; see, for instance, U.S. Pat. Nos. 10,107,796, and 7,407,756.

### (III) PHARMACEUTICAL COMPOSITIONS

As used herein, the term “pharmaceutical composition” refers to a composition in which an active agent (such as a recombinant AAV virion capable of expressing human ELP1 in a target cell) is formulated together with one or more pharmaceutically acceptable carriers.

Pharmaceutical compositions suitable for internal use include sterile aqueous solutions or dispersions and sterile powders for the extemporaneous preparation of sterile injectable solutions or dispersion. For many modes of administration, including for instance intravenous and ocular administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants such as polysorbates (Tween™), sodium dodecyl sulfate (sodium lauryl sulfate), lauryl dimethyl amine oxide, cetyltrimethylammonium bromide (CTAB), polvethoxylated alcohols, polyoxyethylene sorbitan, octoxvnol (Triton X100™), NN-dimethvldodecylamine-N-oxide, hexadecyltrimethylammonium bromide (1H4TAB), polyoxyl 10 lauryl ether, Brij 721™, bile salts (sodium deoxycholate, sodium cholate), pluronic acids (F-68, F-127), polyoxyl castor oil (Cremophor™) nonylphenol ethoxylate (Tergitol™). cyclodextrins and, ethylbenzethonium chloride (Hy Amine™). Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile filtered solution thereof.

The vector or recombinant viruses (virions) can be incorporated into pharmaceutical compositions for administration to mammalian patients, particularly humans. The vector or virions can be formulated in nontoxic, inert, pharmaceutically acceptable aqueous carriers, preferably at a pH ranging from 3 to 8, more preferably ranging from 6 to 8, most preferably ranging from 6.8 to 7.2. Such sterile compositions will include the vector or virion containing the nucleic acid encoding the therapeutic molecule dissolved in an aqueous buffer having an acceptable upon reconstitution.

In some aspects, the pharmaceutical compositions provided herein include a therapeutically effective amount of a vector or virion in admixture with a pharmaceutical acceptable carrier and/or excipient, for example saline, phosphate buffered saline, phosphate and amino acids, polymers, polyols, sugar, buffers, preservatives, and other proteins. Exemplary amino acids, polymers and sugars and the like are octylphenoxy polvethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least 14 months at −60° C.

Such formulations include a pharmaceutically and/or physiologically acceptable vehicle, diluent, carrier, or excipient, such as buffered saline or other buffers, e.g., HEPES, to maintain physiologic pH. For a discussion of such components and their formulation, see, generally, Gennaro, A. E., Remington: The Science and Practice of Pharmacy, Lippincott Williams & Wilkins Publishers; 2003 or latest edition; see also, WO 2000/015822A1. If the preparation is to be stored for long periods, it may be frozen, for example, in the presence of glycerol.

In some aspects, the pharmaceutical composition provided herein includes a buffer, such as phosphate buffered saline (PBS) or sodium phosphate/sodium sulfate, tris buffer, glycine buffer, sterile water, and other buffers known to the ordinarily skilled artisan such as those described by Good et al. (Biochemistry 5:467, 1966). In some aspects, pharmaceutical composition contains sodium phosphate, sodium chloride and orbital. In some aspects, the pharmaceutical composition contains 10 mM sodium phosphate, 350 mM sodium chloride, and 5% (v/v) sorbitol. The pH of the buffer in which the pharmaceutical composition including the mCoChop contained in the adenoviral vector delivery system, may be in the range of 6.5 to 7.75, 6.5 to 7.5, 6.8 to 7.4, or 6.8 to 7.2. In some aspects, the pharmaceutical composition provided herein includes substances that increase the viscosity of the suspension, such as sodium carboxymethyl cellulose, sorbitol, or dextran, in the amount of about 1-10 percent, such as 1, 2, 3, 4, 5, 6, 7, 8, 9, or 10 percent (v/v). Preferably the sorbitol is about 3-6% (v/v), most preferably the sorbitol is about 5%. (v/v).

Before administration the pharmaceutical composition can be tested to ensure it is free of components used during the production, e.g., culture components, host cell protein, host cell DNA, and plasmid DNA; and substantially free of mycoplasma, endotoxin, and microbial contamination. Preferably, the pharmaceutical composition has less than 10, 5, 3, 2, or I CFU/swab. Most preferably, the pharmaceutical composition has 0 CFU/swab. The endotoxin level in the pharmaceutical composition is less than 20 EU/mL, less than 10 EU/mL, or less than 5 EU/mL. The pharmaceutical composition must have sufficiently full capsid prior to administration. The pharmaceutical composition has at least 50%, at least 60%, at least 70%, at least 80% or greater full capsids.

Suitable routes of administration for targeting ocular cells include, for example, intravitreal, intraocular, or subretinal injection. Preferably, the route of administration is by intravitreal injection. All retinal neurons, including retinal ganglion cells, bipolar cells, horizontal cells, amacrine cells, and photoreceptor cells are known to be reasonably well-accessible to intravitreal injection as disclosed herein. Intravitreal and/or subretinal injection may provide the necessary access to the bipolar cells, especially in circumstances in which the photoreceptor cell layer is absent due to degeneration. Other central nervous system administration routes may be appropriate when other CNS-involved aspects of FD are being treated. See, for instance, U.S. Pat. Nos. 11,197,937 and 10,982,228.

Other routes of administration are more appropriate when peripheral nervous system (PNS) symptoms are the target, for instance the target of treatment. These include local injection (such as spinal injection), intravenous injection, intrathecal administration, and so forth.

In one embodiment, the constructs or nucleic acid expression vectors described herein are packaged in adenoviral vectors for transgene delivery. An effective amount of rAAV virions carrying a hELP1 transgene under the control of a promoter is preferably in the range of between about 10¹⁰ to about 10¹³ rAAV infectious units in a volume of between about 150 and about 800 μl per injection. The rAAV infectious units can be measured according to Mclaughlin et al. (J. Viral. 62:1963, 1988). In embodiments, the effective amount is between about 10¹⁰ and about 10¹² rAAV infectious units and the injection volume is preferably between about 250 and about 500 μl. Other dosages and volumes, preferably within these ranges but possibly outside them, may be selected by the treating professional, considering the physical state of the mammalian subject (in embodiments, a human) who is being treated, including for instance age, weight, general health, and the nature and severity of the particular disorder (e.g., ocular disorder) being treated.

In some cases, the viral vector of the disclosure may be measured as pfu (plaque forming units). In some cases, the pfu of recombinant virus, or viral vector of the compositions and methods of the disclosure may be about 10⁸ to about 5×10¹⁰ pfu. In some cases, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some cases, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu.

In some cases, the viral vector of the disclosure may be measured as vector genomes. In some cases, recombinant viruses of this disclosure are 1×10¹⁰ to 3×10¹² vector genomes. In some cases, recombinant viruses of this disclosure are 1×10⁹ to 3×10¹³ vector genomes. In some cases, recombinant viruses of this disclosure are 1×10⁸ to 3×10¹⁴ vector genomes. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some cases, the viral vector of the disclosure may be measured using a multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI.

It may also be desirable to administer additional doses (“boosters”) of the present nucleic acid(s) or rAAV compositions. For example, depending upon the duration of the transgene expression within the target cell(s) (such as ocular target cell(s)), a second administration may be administered after 6 months or yearly, and may be similarly repeated. Neutralizing antibodies to AAV are not expected to be generated in view of the routes and doses used, thereby permitting repeat administration rounds. The need for such additional doses can be monitored by the treating professional using, for example, well-known electrophysiological and other retinal and visual function tests and visual behavior tests. The treating professional will be able to select the appropriate tests by applying routine skill in the art. It may be desirable to inject larger volumes of the composition in either single or multiple doses to further improve the relevant outcome parameters.

As used herein, the term “pharmaceutically acceptable,” as applied to one or more, or all, component(s) for formulation of a composition as disclosed herein, means that each component must be compatible with the other ingredients of the composition and not unacceptably deleterious to the recipient thereof.

Generally, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

### (VI) KITS

Compositions and reagents useful for the present disclosure may be packaged in kits to facilitate application of the present disclosure. In some aspects, the present disclosure provides for a kit including a recombinant nucleic acid of the disclosure. In some aspects, the kit includes a recombinant virus of the disclosure. Optionally, the kit may also include instructions for use of the recombinant virus. The instructions could be in any desired form, including but not limited to, printed on a kit insert, printed on one or more containers, as well as electronically stored instructions provided on an electronic storage medium, such as a computer readable storage medium. Also optionally included is a software package on a computer readable storage medium that permits the user to integrate the information and calculate a control dose. In another aspect, the present disclosure provides a kit including the pharmaceutical compositions provided herein. In yet another aspect, the disclosure provides kits for instance for administration to a subject, which may be for the treatment of disease(s).

In one aspect, a kit includes: (a) a recombinant virus provided herein, and (b) instructions to administer to cells or an individual a therapeutically effective amount of the recombinant virus. In some aspects, the kit may include pharmaceutically acceptable salts or solutions for administering the recombinant virus. Optionally, the kit can further include instructions for suitable operational parameters in the form of a label or a separate insert. For example, the kit may have standard instructions informing a physician or laboratory technician to prepare a dose of recombinant virus.

Optionally, the kit may further include a standard or control information so that a patient sample can be compared with the control information standard to determine if the test amount of recombinant virus is a therapeutic amount Optionally, the kit could further include devices for administration, such as a syringe, filter needle, extension tubing, cannula, and subretinal injector.

Recombinant viruses may be generated by any suitable means. The methods and compositions of the disclosure provide for generation of recombinant viruses through various means, including the use of transgenic cells, which may include mammalian cells, insect cells, animal cells, or fungal cells.

For example, in some aspects, recombinant viruses may be generated through transfection of insect cells via recombinant baculovirus. In some cases, recombinant baculovirus may be generated as an intermediate, whereby the baculovirus may contain sequences necessary for the generation of other viruses such as AAV or rAAV2 viruses. In some cases, one or more baculoviruses may be used in the generation of recombinant viruses used for the composition and methods of administration and/or treatment of this disclosure. In some cases, insect cells such as Sf9, High-Five, or Sf21 cell lines may be used. In some cases, cell lines may be generated using transient methods (i.e., infection with not stably integrated transgenes). In other cases, cell lines may be generated through the generation of stable cell lines (i.e., infection with transgenes stably integrated into the host cell genome.) In other aspects, the pharmaceutical composition provided herein is manufactured using adherent human embryonic kidney 293 (HEK293) cells. In an alternative aspect, the pharmaceutical composition provided herein is manufactured using suspension-adapted HEK293 cells. In another aspect, the pharmaceutical composition provided herein is manufactured using the baculovirus expression system (BYES) in insect cells. In some aspects, the vector is produced using herpes-helper virus. In some aspects, the vector is produced using producer-clone methods. In some aspects, the vector is produced using Ad-AAV.

Generally, any suitable method may be used in the biochemical purification of recombinant viruses for use in a pharmaceutical composition as described herein. Recombinant viruses may be harvested directly from cells, or from the culture media surrounding host cells. Viruses may be purified using various biochemical means, such as gel filtration, filtration, chromatography, affinity purification, gradient ultracentrifugation, or size exclusion methods. Recombinant virus may be tested for content (i.e., identity), purity, or potency (i.e., activity) using any suitable means, before formulation into a pharmaceutical composition. Method may include but are not limited to immunoassays, ELISA, SDS-PAGE, western blot, Northern blot, Southern blot, or PCR, HUVEC assays, and the like.

### (V) REPRESENTATIVE DEFINITIONS

In various aspects, vectors of the disclosure are used to increase, establish, and/or maintain the expression of human ELP1 protein. The terms “polypeptide” and “protein” are used interchangeably herein to refer to a polymer of amino acid residues and to variants and synthetic analogs of the same. Thus, these terms apply to amino acid polymers in which one or more amino acid residues are synthetic non-naturally occurring amino acids, such as a chemical analog of a corresponding naturally occurring amino acid, as well as to naturally-occurring amino acid polymers.

Particular aspects of the disclosure also include polypeptide “variants.” The recitation polypeptide “variant” refers to polypeptides that are distinguished from a reference polypeptide by the addition, deletion, truncations, and/or substitution of at least one amino acid residue, and that retains a biological activity of the reference polypeptide. In certain aspects, a polypeptide variant is distinguished from a reference polypeptide by one or more substitutions, which may be conservative or non-conservative, as known in the art.

In certain aspects, a variant polypeptide includes an amino acid sequence having at least about 50%, 55%, 60%, 65%, 70%, 75%, 80%, 85%, 90%, 91%, 92%, 93%, 94%, 95%, 96%, 97%, 98%, 99% or more sequence identity or similarity to a corresponding sequence of a reference polypeptide. In certain aspects, amino acid additions or deletions occur at the C-terminal end and/or the N-terminal end of the reference polypeptide.

A variant may have 50%, 55%, 60%, 65%, 70%, 75%, 80%, 85%, 90%, 95%, 96%, 97%, 98%, or 99% sequence identity to the nucleic acid sequences described herein. The term “% identity,” in the context of two or more nucleic acid or polypeptide sequences, refers to two or more sequences or subsequences that are the same or have a specified percentage of amino acid residues or nucleotides that are the same, when compared and aligned for maximum correspondence, as measured using one of the following sequence comparison algorithms or by visual inspection. For example, % identity is relative to the entire length of the coding regions of the sequences being compared.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, WI) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte & Doolittle, J. Mol. Biol. 157 (1), 105-32, 1982). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure, it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2 M NaH₂PO₄; 0.02 M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity is determined using search algorithms such as BLAST and PSI-BLAST (Altschul et al., 1990, J. Mol. Biol. 215:3, 403-410; Altschul et al., 1997, Nucleic Acids Res. 25:17, 3389-402).

A “host cell” includes cells transfected, infected, or transduced in vivo, ex vivo, or in vitro with a recombinant vector or a polynucleotide of the disclosure. Host cells may include packaging cells, producer cells, and cells infected with viral vectors. In particular aspects, host cells infected with a viral vector of the disclosure are administered to a subject in need of therapy. In certain aspects, the term “target cell” is used interchangeably with the host cell and refers to transfected, infected, or transduced cells of a desired cell type.

By “maintain,” or “preserve,” or “maintenance,” or “no change,” or “no substantial change,” or “no substantial decrease” refers generally to a physiological response that is comparable to a response caused by either vehicle, a control molecule/composition, or the response in a particular cell lineage. A comparable response is one that is not significantly different or measurably different from the reference response.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Preferably, the subject is a human. A “subject in need thereof” is a subject suffering from or at risk of developing or suffering from an ocular disease or disorder. A subject at risk of developing or suffering from an ocular disease or disorder can be diagnosed by a physician or ocular specialist using routine methods in the art. For instance, a subject can be identified by testing for one or more of the mutations associated with FD disease.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms, and/or burden of a disease or condition prior to onset or recurrence of the disease or condition.

As used herein, the term “amount” refers to “an amount effective” or “an effective amount” of a virus or transduced therapeutic cell to achieve a beneficial or desired prophylactic or therapeutic result, including clinical results.

A “prophylactically effective amount” refers to an amount of a virus or transduced therapeutic cell effective to achieve the desired prophylactic result. Typically, but not necessarily, since a prophylactic dose is used in subjects prior to or at an earlier stage of disease, the prophylactically effective amount is less than the therapeutically effective amount.

As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

A “therapeutically effective amount” of a virus or transduced therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient).

As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

As used herein, “expression” refers individually and/or cumulatively to one or more biological process that result in production from a nucleic acid sequence of an encoded agent, such as a protein. Expression specifically includes either or both of transcription and translation.

As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment (or sub-sequence) of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

As used herein, the term “gene product” or “expression product” generally refers to an RNA transcribed from the gene (pre- and/or post-processing) or a polypeptide (pre- and/or post-modification) encoded by an RNA transcribed from the gene.

As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. This includes any beneficial or desirable effect on the symptoms or pathology of a disease or pathological condition and may include even minimal reductions in one or more measurable markers of the disease or condition being treated. In some embodiments, such treatment can be of a subject who does not exhibit signs of the relevant disease, disorder, or condition and/or of a subject who exhibits only early signs of the disease, disorder, or condition. “Treatment” does not necessarily indicate complete eradication or cure of the disease or condition, or associated symptoms thereof.

Treatment can involve optionally either the reduction or amelioration of symptoms of the disease or condition, or the delaying of the progression of the disease or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

“Administration” is a broader term than treatment, at least in that it does not require that the subject to which a composition or compounds is being administered need not have been diagnosed with a disease or pathology.

As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (VI) EXEMPLARY EMBODIMENTS

1. An isolated nucleic acid molecule including: a polynucleotide including a nucleic acid sequence encoding a human ELP1 (hELP1) protein; a promoter sequence arranged to promote expression of the human ELP1 protein in a mammalian cell; and a post-transcriptional regulatory element.

2. The isolated nucleic acid molecule of embodiment 1, wherein the promoter sequence promotes expression of the ELP1 protein in a mammalian neuron.

3. The isolated nucleic acid molecule of embodiment 2, wherein the promoter sequence promotes expression of the ELP1 protein in a central nervous system (CNS) neuron.

4. The isolated nucleic acid molecule of embodiment 3, wherein the promoter sequence promotes expression of the ELP1 protein in a retinal neuron.

5. The isolated nucleic acid molecule of any one of embodiments 1-4, wherein the isolated nucleic acid molecule includes a recombinant AAV2 vector, and the polynucleotide is flanked by AAV ITR sequences.

6. The isolated nucleic acid molecule of embodiment 1, wherein the encoded human ELP1 protein includes an amino acid sequence at least 95% identical to the sequence of SEQ ID NO: 7.

7. The isolated nucleic acid molecule of embodiment 1, wherein the nucleotide sequence encoding the human ELP1 protein has a sequence at least 95% identical to the sequence of SEQ ID NO: 6.

8. The isolated nucleic acid molecule of embodiment 1, wherein the nucleotide sequence encoding the human ELP1 protein includes the sequence of SEQ ID NO: 6.

9. The isolated nucleic acid molecule of embodiment 5, wherein the promoter sequence is no more than about 300 nucleotides long.

10. The isolated nucleic acid molecule of embodiment 1, wherein the promoter sequence is from a small nuclear RNA protein (SNRPA).

11. The isolated nucleic acid molecule of any of embodiments 1-10, wherein the promoter sequence is a small nuclear RNA U1 promoter.

12. The nucleic acid molecule of embodiment 11, wherein the promoter sequence is a murine or human Ula promoter.

13. The nucleic acid molecule of embodiment 11, wherein the promoter has a sequence including or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell.

14. The nucleic acid molecule of embodiment 1, wherein the polynucleotide molecule includes: a nucleotide sequence at least 95% identical to SEQ ID NO: 6 and encoding a functional human ELP1 protein; and a Ula promoter sequence arranged to promote expression of the human ELP1 protein.

15. The nucleic acid molecule of embodiment 1, wherein the polynucleotide molecule includes: a nucleotide sequence identical to SEQ ID NO: 6; a Ula promoter sequence having the sequence SEQ ID NO: 4 arranged to promote expression of the human ELP1 protein; or both.

16. The nucleic acid molecule of embodiment 13, wherein the polynucleotide molecule has a sequence at least 95% identical to SEQ ID NO: 1.

17. The nucleic acid molecule of embodiment 15, wherein the polynucleotide molecule has a sequence identical to SEQ ID NO: 1.

18. A recombinant AAV (rAAV) virion, including: an AAV2 capsid; and the nucleic acid molecule of any one of embodiments 1-17.

19. A recombinant AAV (rAAV) virion including: an AAV2 capsid; and a recombinant polynucleotide including a nucleic acid sequence encoding human ELP1 operably linked to a promoter that promotes expression of the human ELP1 in retinal neurons, flanked by AAV ITR sequences.

20. A recombinant adeno-associated serotype 2 (rAAV2) virion including: a rAAV2 vector polynucleotide; and, contained therein: a human ELP1 gene nucleic acid sequence (hELP1); a U1a promoter sequence arranged to promote expression of the human ELP1 protein; and a post-transcriptional regulatory element.

21. The rAAV of embodiment 20, in which the polynucleotide includes a nucleotide sequence functionally equivalent to the nucleotide sequence of SEQ ID NO: 1 (rAAV2.U1a.hELP1).

22. A pharmaceutical composition including the rAAV of any one of embodiments 18-21, formulated for administration to a mammalian subject.

23. The pharmaceutical composition of embodiment 22, formulated for ocular administration.

24. Use of the rAAV of any one of embodiments 18-21, or the composition of embodiment 22 or 23, to express hELP1 protein in a cell the genome of which contains a mutated hELP1 gene.

25. The use of embodiment 24, wherein the cell is a central nervous system cell or a peripheral nervous system cell of a mammal.

26. The use of embodiment 24, wherein the cell is a neuron.

27. A method of increasing ELP1 expression in retinal cells of a mammalian subject in need thereof, the method including: administering to the mammalian subject a pharmaceutical formulation including an AAV2-hELP1 vector including: a AAV2 capsid, and a recombinant polynucleotide including: a nucleic acid sequence encoding human ELP1 operably linked to a promoter that promotes expression of the human ELP1 in retinal neurons, flanked by AAV ITR sequences; wherein the AAV2-hELP1 vector is delivered to at least one target site in the subject.

28. The method of embodiment 27, wherein one or more retinal cells of the mammalian subject express a mutant ELP1 protein.

29. The method of embodiment 27, wherein one or more retinal cells of the mammalian subject exhibit reduced expression of ELP1 as compared to a normal subject.

30. The method of embodiment 27, wherein one or more retinal cells of the mammalian subject express an ELP1 protein with reduced functionality as compared to a normal subject.

31. The method of embodiment 27, wherein administering the pharmaceutical formulation to the mammalian subject results in enhanced survival and/or functionality of retinal cells of the mammalian subject.

32. A method of increasing ELP1 expression in cells of a mammalian subject in need thereof, the method including: providing a pharmaceutical formulation including an AAV2-hELP1 vector including: a AAV2 capsid, and a recombinant DNA insert including a nucleic acid sequence encoding human ELP1; and delivering the pharmaceutical formulation to at least one target site in the central nervous system (CNS) or in the peripheral nervous system (PNS) of the subject.

33. The method of embodiment 32, wherein one or more retinal cells of the mammalian subject express a mutant ELP1 protein.

34. The method of embodiment 32, wherein one or more retinal cells of the mammalian subject exhibit reduced expression of ELP1 as compared to a normal subject.

35. The method of embodiment 32, wherein one or more retinal cells of the mammalian subject express an ELP1 protein with reduced functionality as compared to a normal subject.

36. The method of embodiment 32, wherein administering the pharmaceutical formulation to the mammalian subject results in enhanced survival and/or functionality of retinal cells of the mammalian subject.

37. The method of embodiment 27 or 32, wherein the target site includes at least one eye of the subject.

38. The method of embodiment 37, wherein the pharmaceutical formulation is administered to the eye of the mammalian subject by an intravitreal, suprachoroidal, subretinal, or intraocular route, optionally by injection.

39. The method of embodiment 37, wherein the method includes delivering to the subject a dose of: 1×10⁹-1×10¹² viral genomes (vg)/eye; 1×10¹⁰-1×10¹¹ vg/eye; or 1×10⁹-1×10¹⁰ vg/eye.

40. The method of embodiment 27 or embodiment 32, wherein treating ELP1 deficiency in the mammalian subject reduces or prevents death of a neuron in the CNS or in the PNS of the mammalian subject.

41. The method of embodiment 27 or embodiment 32, wherein the mammalian subject is a human subject.

42. The method of embodiment 41, wherein the human subject is diagnosed with familial dysautonomia (FD).

43. The method of embodiment 42, wherein the human subject has a mutated ELP1 gene.

44. The method of embodiment 27 or embodiment 32, wherein the nucleic acid sequence encoding hELP1 is an hELP1 cDNA.

45. The method of embodiment 32, wherein the recombinant DNA insert includes from 5′ to 3′: a first inverted terminal repeat (ITR), a small nuclear RNA U1a promoter, the nucleic acid sequence encoding functional human ELP1, a poly A signal sequence, and a second ITR.

46. The method of embodiment 32, wherein the recombinant DNA insert includes: a U1a promoter including or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell, and a nucleic acid sequence at least 95% identical to SEQ ID NO: 6.

47. The method of embodiment 27 or embodiment 32, wherein the AAV2-hELP1 vector includes the nucleotide sequence of SEQ ID NO: 1.

48. The method of embodiment 27 or embodiment 32, wherein the pharmaceutical formulation is delivered by injection or infusion.

49. The method of embodiment 27 or 32, wherein the pharmaceutical formulation is delivered to eye of the subject by an intravitreal, suprachoroidal, subretinal, or intraocular route, optionally by injection.

50. A method of increasing ELP1 protein expression in a central nervous system (CNS) cell or a peripheral nervous system (PNS) cell of a mammalian subject, including administering to the CNS or the PNS of the mammalian subject a viral expression vector including: a human ELP1-encoding nucleic acid sequence; and a small nuclear RNA Ula promoter arranged to promote expression of the human ELP1 protein in a cell of the CNS or the PNS.

51. The method of embodiment 50, wherein one or more CNS cells or PNS cells of the mammalian subject express a mutant ELP1 protein.

52. The method of embodiment 50, wherein one or more CNS cells or PNS cells of the mammalian subject exhibit reduced expression of ELP1 as compared to a normal subject.

53. The method of embodiment 50, wherein one or CNS cells or PNS cells of the mammalian subject express an ELP1 protein with reduced functionality as compared to a normal subject.

54. The method of embodiment 50, wherein administering the viral expression vector results in enhanced survival and/or functionality of retinal cells of the mammalian subject.

55. The method of embodiment 50, wherein the viral expression vector is an AAV2 viral expression vector.

56. The method of embodiment 55, wherein the human ELP1-encoding nucleic acid has a sequence at least 95% identical to the sequence of SEQ ID NO: 6.

57. The method of embodiment 56, wherein the human ELP1-encoding nucleic includes the sequence of SEQ ID NO: 6.

58. The method of embodiment 55 or embodiment 56, wherein the encoded human ELP1 protein has a sequence at least 95% identical to the sequence of SEQ ID NO: 7.

59. The method of embodiment 58, wherein the encoded human ELP1 protein includes the sequence of SEQ ID NO: 7.

60. The method of embodiment 50, wherein the promoter is a ubiquitous promoter or a retinal cell-specific promoter.

61. The method of embodiment 50, wherein the promoter is a murine Ula promoter or a human Ula promoter.

62. The method of embodiment 61, wherein the promoter has a sequence including or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell.

63. The method of embodiment 50, wherein the viral vector includes an adeno-associated virus serotype 2 (AAV2) or an adeno-associated virus serotype 9 (AAV9) vector.

64. The method of embodiment 63, wherein the AAV2 vector includes AAV2-U1a-Elp1.

65. A method of increasing ELP1 protein level in a central nervous system (CNS) cell or a peripheral nervous system (PNS) cell of a mammal, essentially as described herein.

66. A recombinant AAV (rAAV) virion, or an ELP1-expressing rAAV vector, essentially as described herein.

### (VII) EXPERIMENTAL EXAMPLE(S)

### Example 1: Reduction of Retinal Ganglion Cell Death in Mouse Models of Familial Dysautonomia Using AAV-Mediated Gene Therapy

Familial dysautonomia (FD) is a rare neurodevelopmental and neurodegenerative disease caused by a splicing mutation in the Elongator Acetyltransferase Complex Subunit 1 (ELP1) gene. The reduction in ELP1 mRNA and protein leads to the death of retinal ganglion cells (RGCs) and visual impairment in all FD patients. Currently, patient symptoms are managed, but there is no treatment for the disease. To test the hypothesis that restoring levels of Elp1 would thwart the death of RGCs in FD, the effectiveness of a therapeutic strategy for rescuing RGCs was tested. Using a mouse model, mice were treated with an intravitreal injection of an AAV2 (highly tropic for RGCs) engineered to express a wildtype copy of ELP1 (Cao et al., Gene Ther, 26 (3-4): 109-120, 2019; Nieuwenhuis et al., Gene Ther, 30 (6): 503-519, 2023; Wassmer et al., Invest Ophthalmol Vis Sci, 61 (8): 49, 2020) (FIGS. 1A, 1B). This example provides proof-of-concept data that gene replacement therapy effectively reduces the death of RGCs in mouse models for FD and provide pre-clinical data foundation for translation to FD patients. At least some of the material described in this Example was published as Schultz et al. (Sci Rep 13, 18600, 2023) including supplementary information, available online May 24, 2023, and in print Oct. 30, 2023 (doi.org/10.1038/s41598-023-45376-w).

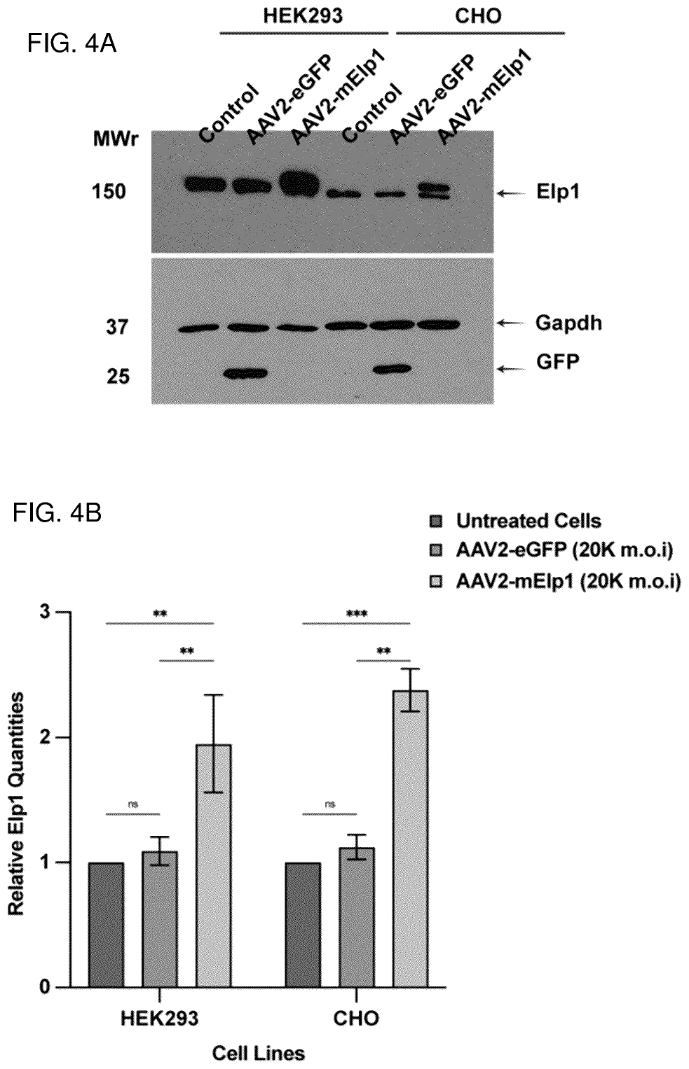

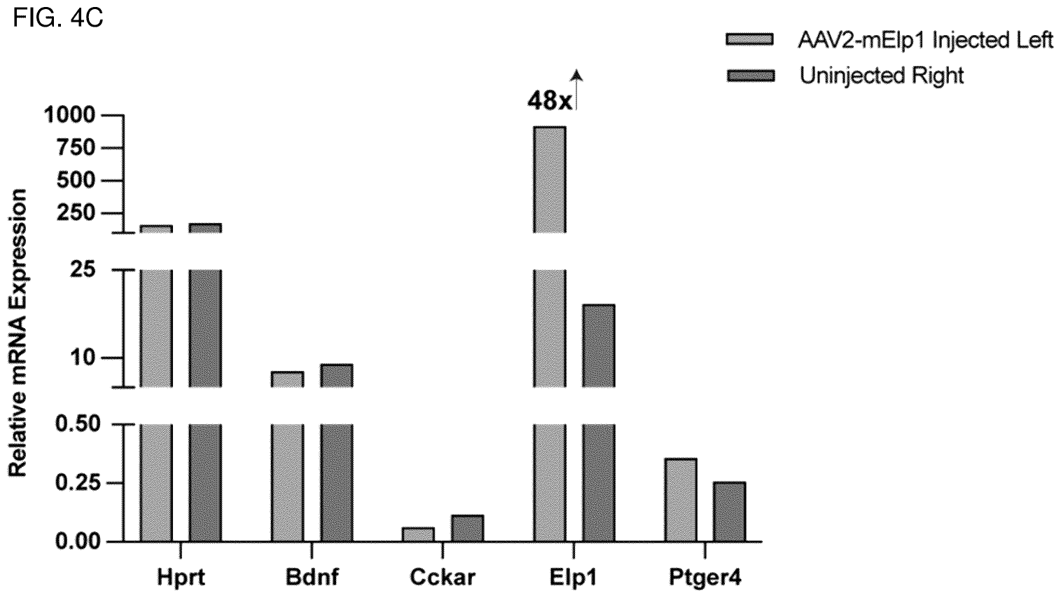

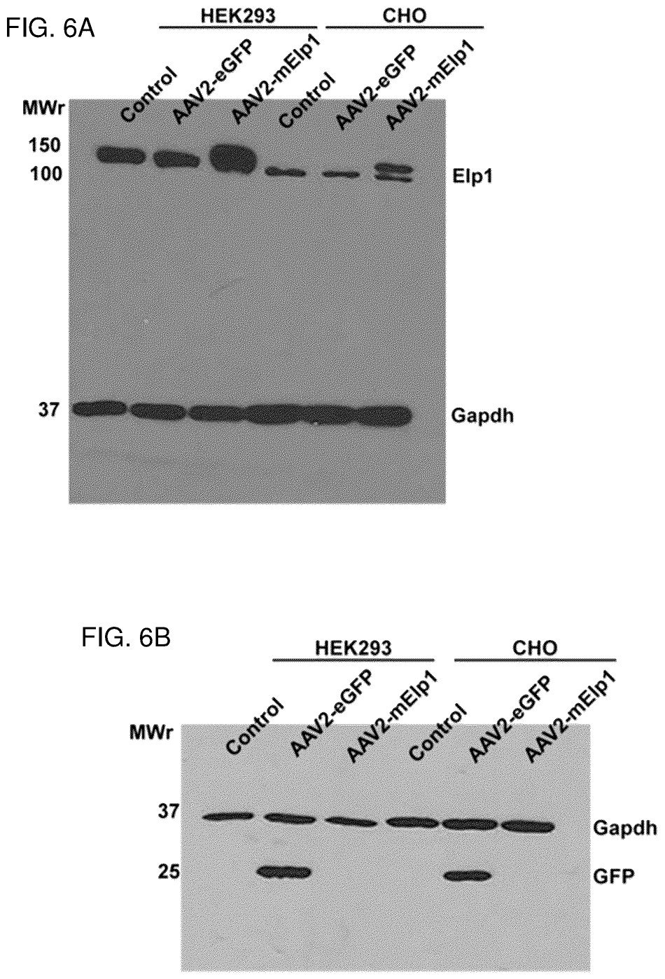

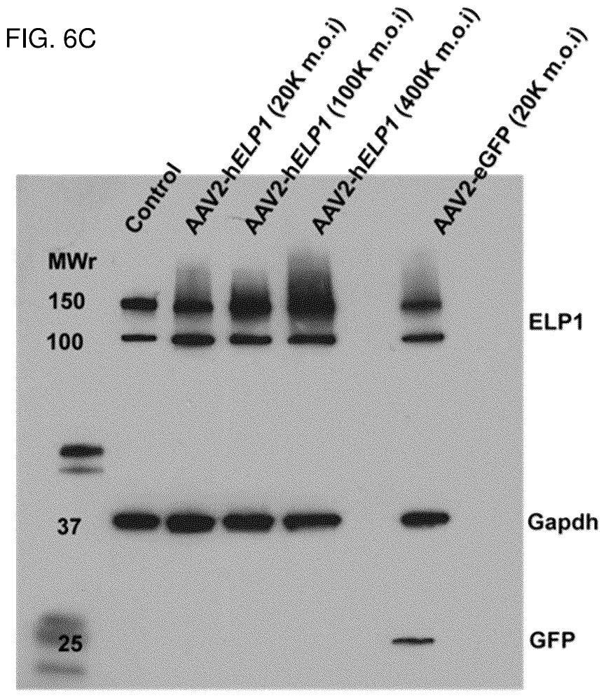

To test whether a gene replacement strategy could prevent the death of RGCs in a previously established Pax6-cre;Elp1^{loxp/loxp} mouse model (Ueki et al., Dis Model Mech, 11 (7), 2018), two AAV2 vectors were generated, one expressing the murine Elp1 gene (mElp1) and the other expressing eGFP, both driven by the Ula promoter (see Methods). It was first determined whether the AAV2-U1a-mElp1 could drive the expression of Elp1 in the mouse retina and increase Elp1 protein expression in vitro (FIGS. 4A-4C, 6A-6C).

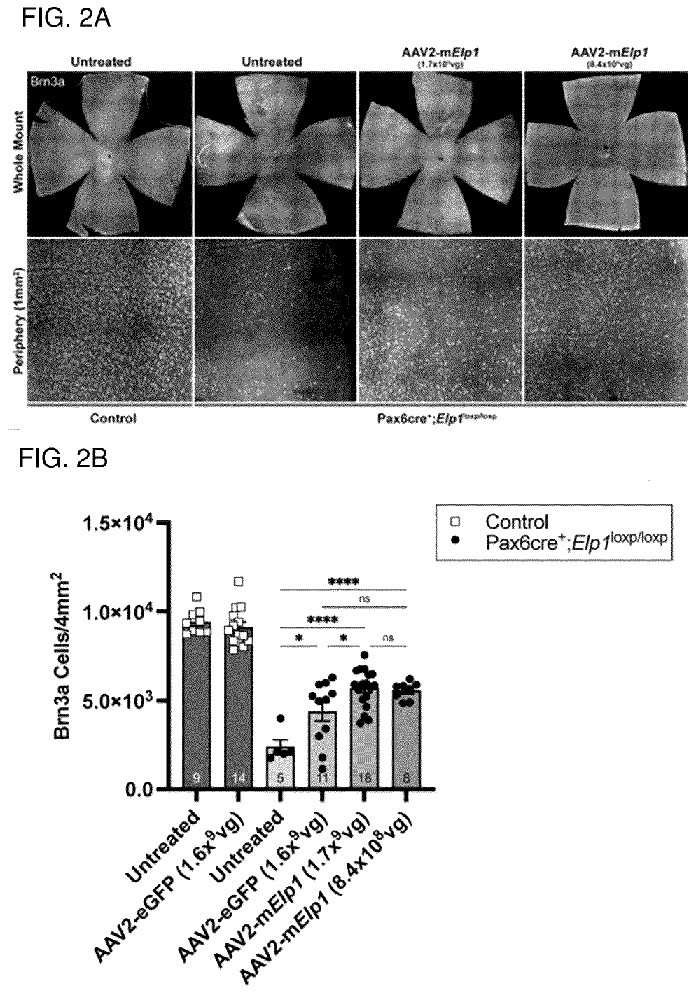

Next, cohorts of Pax6cre; Elp1^{loxp/loxp} mice received intravitreal injections of AAV2-U1a-mElp1 or control (eGFP) vector at postnatal day 21 (P21) before any significant loss of RGCs is observed (Ueki et al., Dis Model Mech, 11 (7), 2018). Three months following injection, RGCs were quantified by flat-mount analysis (FIG. 2A). This time point was selected since prior work established that, by this age, 40-60% of RGCs have died (Ueki et al., Dis Model Mech, 11 (7), 2018). A significant response was observed, with a 137% increase in RGC number (Mean: 5696±252, p<0.0001) with the higher AAV2-U1a-mElp1 vector dose (1.7×10⁹ vg/eye) and a 131% increase in RGC number (Mean: 5563±172, p<0.0001) with the lower AAV2-U1a-mElp1 vector dose (8.4×10⁸ vg/eye) in Pax6cre; Elp1^{loxp/loxp} retinae compared to untreated Pax6cre; Elp1^{floxp/floxp} retinae (Mean: 2404±405) (FIG. 2B). Interestingly, eyes injected with the control AAV2-U1a-eGFP showed an 82% increase in RGCs (Mean: 4386±534) compared to the untreated Pax6cre; Elp1^{loxp/loxp} (FIG. 2B). However, the mElp1-virus significantly increased RGC numbers by 30% (p=0.04) above the eGFP-injected eyes. This suggests that (1) injection of a virus in a dose-dependent manner alone induces a protective response in the retina, most likely by stimulating a wound response (Steinberg, Curr Opin Neurobiol, 4 (4): 515-24, 1994; Lau et al., Invest Ophthalmol Vis Sci, 41 (11): 3622-33, 2000; Cao et al., Exp Eye Res, 65 (2): 241-8, 1997), but (2) that the Elp1 virus increased RGCs significantly above the virus-alone-induced protective response. These data provide promising evidence that gene replacement therapy, with further optimization, could be used to ameliorate RGC death in FD.

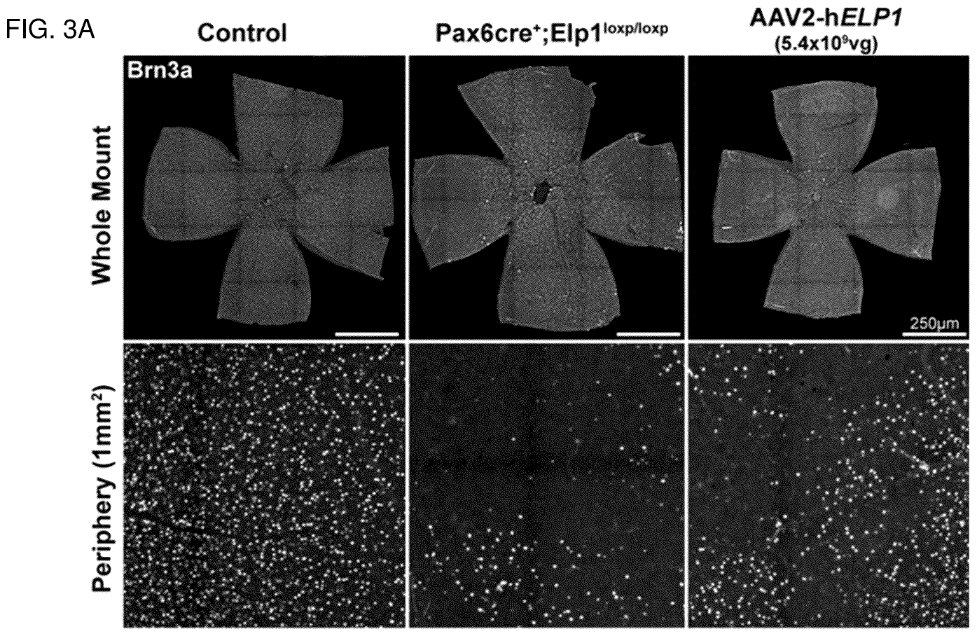

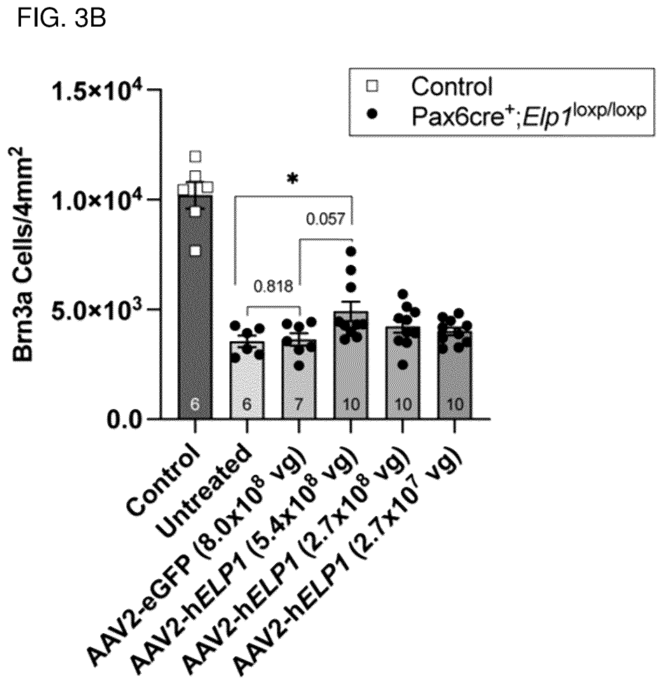

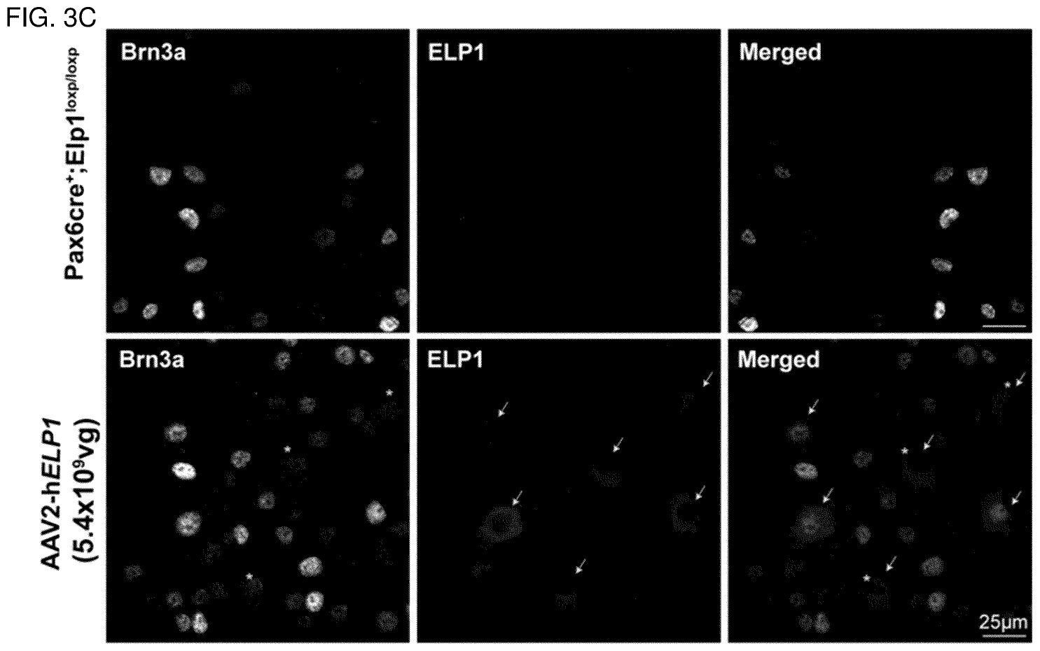

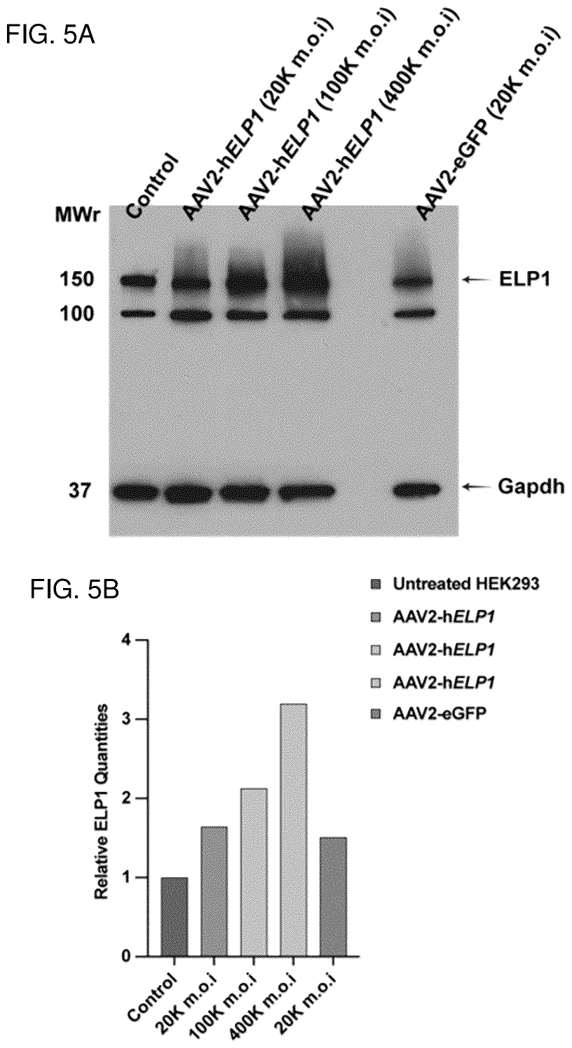

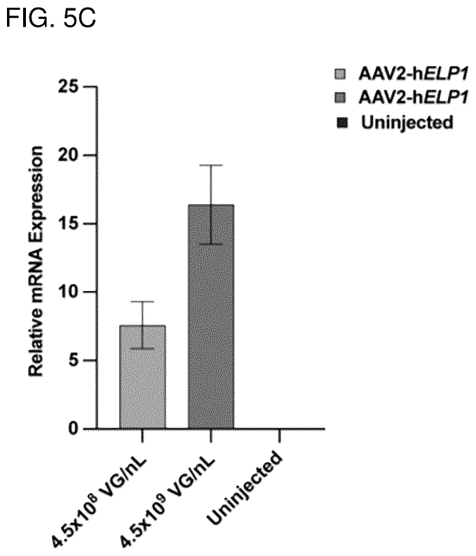

Based on these data, an AAV2 vector driving the expression of the human ELP1 gene (hELP1) was generated. Again, it was first confirmed that the AAV2-U1a-hELP1 could increase ELP1 protein in vitro (FIGS. 5A, 5B) and drive ELP1 mRNA expression in wild-type mouse retinas in vivo (FIG. 5C). Subsequently, Pax6cre;Elp1^{loxp/loxp} and littermate controls were intravitreally injected with three different viral titers of the human ELP1 virus. All mice received bilateral injections at P21, and RGCs were quantified three months later (FIG. 3A). There was a significant increase in RGC survival in the Pax6cre; Elp1^{loxp/loxp} retinae that received the highest vector dose (5.4×10⁸ vg/eye), showing a 39% increase in RGC number (Mean: 4925±439, p=0.02) (FIG. 3B). There was no significant difference between the uninjected retina and those injected with the AAV2-U1a-eGFP construct, which was used at a lower dose than in the mouse Elp1 virus experiments. To visualize the AAV2-U1a-hELP1 infected RGCs, retinae were double immunolabeled with antibodies to RGCs and human ELP1. In the retinae infected with AAV2-U1a-hELP1, approximately 26% of RGCs expressed the human ELP1 protein (FIG. 3C), visually confirming that driving ELP1 expression can efficiently produce human ELP1 protein.

This work demonstrated that an intravitreally-delivered RGC-specific gene transfer can prevent the progressive death of RGCs in models of FD. This is the first gene therapy study in an FD model and lays the foundation for a translational path for mitigating RGC loss in FD patients. These data support treatment of FD patients to prevent or reduce RGC degeneration, as most FD patients do not report vision loss until they are teenagers.

Data presented here show that approximately 30% of RGCs were infected with the human ELP1 virus and expressed the human ELP1 protein, which is in the same range as the percentage of RGCs rescued (39%). Moreover, this provides the first evidence that morphology is not disrupted in RGCs expressing virally transduced ELP1, suggesting that overexpression of human ELP1 is not harmful to retinal neurons.

These data justify the further optimization of an ELP1-vector for translation to a clinical setting. Potential paths forward include optimizing the AAV2 capsid properties via chemical modifications or assembling mosaic capsids consisting of different serotypes could improve targeting specificity and tropism to achieve higher transduction efficiency (Byrne et al., JCI Insight, 5 (10), 2020; Frederick et al., Hum Gene Ther, 31 (13-14): 756-774, 2020; Öztürk et al., Elife, 10, 2021). Alternatively, concatamerization of dual AAV vectors (Carvalho et al., Front Neurosci, 11:503, 2017; Dyka et al., Hum Gene Ther, 30 (11): 1361-1370, 2019; Maddalena et al., Mol Ther, 26 (2): 524-541, 2018; McClements et al., Hum Gene Ther, 30 (5): 590-600, 2019; Trapani et al., Methods Mol Biol, 1715:153-175, 2018) can be considered as the ELP1 transgene is large (4 kb), making packaging challenging. This would provide more flexibility in promoter choice to maximize transduction efficiency in RGCs.

FD is a complex developmental and degenerative disease, yet because it is caused by a single splicing-associated point mutation, it is an excellent candidate disease for gene therapy. In summary, the data presented here provide a rationale for translating gene replacement therapy into the clinic to prevent the progressive optic neuropathy that plagues all FD patients.

### Materials and Methods

Animals: All mice were housed in the AALAC-accredited Animal Resource Center at Montana State University. All animal use protocols were approved by the Montana State University Institutional Animal Care and Use Committee (IACUC) (protocol No. 2020-15-IA; Bozeman, MT). The study fulfilled the ARRIVE guidelines, and all experiments complied with relevant guidelines and regulations. The generation of the Pax6cre+; Elp1^{flox/flox} CKO is described in Ueki et al., Dis. Model Mech. 11 (7), 2018. Briefly, retina-specific Elp1 CKO mice were generated by crossing Elp1 floxed (International Knockout Mouse Consortium, Wellcome Sanger Institute, UK) and αPax6 promoter-driven Cre (Pax6-Cre) mice (Marquardt et al., Cell, 105 (1): 43-55, 2001), which were a gift from Drs Peter Gruss and Ruth Ashery-Padan. This is a useful mouse model to study the FD optic neuropathy due to the restricted deletion of Elp1 only in retinal neurons with a time course of RGC loss consistent with patients while maintaining a phenotypically healthy mouse.

The sample size for all experiments was based on an A priori power analysis of 90% with an effect size of 2 and a of 0.05 to detect a statistical difference between the experimental and control groups (Ueki et al., Dis Model Mech, 11 (7), 2018). Both male and female mice were used at the ages indicated (E18.5 up to 6 months), and all controls were littermate Pax6cre-; Elp1^{flox/flox} Mice were randomly assigned to a treatment group based on sex and genotype. All mice were housed in a 12-hour light-dark cycle and were fed chow and water ad libitum.

AAV vector design and production: All AAV viruses were made at the Vector Core in the Gene Therapy Center at the University of Massachusetts Medical School. AAV2 was selected based on its reported tropism for mouse retinal ganglion cells and its use and tolerance in the human eye (Bennett et al., The Lancet, 388 (10045): 661-672, 2016; Koilkonda et al., Invest Ophthalmol Vis Sci, 55 (12): 7739-53, 2014; Harvey et al., Mol Cell Neurosci, 21 (1): 141-57, 2002) and preliminary unpublished experiments revealing its higher expression efficiency in retinal ganglion cells in the described mouse line compared to AAV9. The AAV2 was used at vector doses ranging from 8×10⁸ to 1.7×10⁹ vector genomes per eye (vg/eye) per mouse for the mouse Elp1, while the human ELP1 virus was used at doses ranging from 2.7×10⁷ to 5.4×10⁸ vg/eye. Vector doses were based on preliminary experiments showing potential viral toxicity at higher doses and evidence for cell rescue at lower doses (data not shown). Each virus expressed the full-length sequence for either mouse Elp1 (Origene MC202501, NM 026079) or human ELP1 (Origene RC207686), ELP1 (NM_003640), and both were driven by a murine small nuclear RNA promoter (U1a). The murine U1a promoter was selected based on its small size (˜250 bp) and ability to effectively transduce cells in the central nervous system (Buck & Wijnholds, Int J Mol Sci, 21 (12), 2020). It is proposed that the human Ula promoter would work similarly.

Cell culture: Human embryonic kidney cells (HEK293) were gifted from Carolyn Machamer at Johns Hopkins, and the Chinese hamster ovary cell (CHO) cells were gifted from Ira Mellman at Yale University. HEK293 were cultured in Dulbecco's Modified Eagle Medium (DMEM, Gibco #12430-054) supplemented with 10% fetal bovine serum (FBS, Gibco #16000-036), 100 U/ml penicillin/100 μg/ml streptomycin (Corning, #30-002-CI), and 2 mM L-Glutamine (Gibco, #25030-081). HEK293 cells submitted to American Type Culture Collection (ATCC) for STR profiling were an 88% match for the HEK293 cell line, CRL-1573.

In vitro AAV transduction: HEK293 cells were seeded onto a 12-well plate at a density of 2.5×10⁴ cells/well for 24 hr. Two viral solutions were diluted (1) AAV2-U1a-hELP1 stock to 2.7×10⁹ VG/ml in DMEM and (2) AAV2-U1a-eGFP stock to 8.8×10⁹ VG/ml in DMEM. The ELP1 solution was serially diluted to three different multiplicities of infection (m.o.i.) at 20K, 100K, and 400K. M.O.I. was calculated by dividing the initial seeding density by the total viral particles. At 24 hours, the complete medium was removed and supplemented with the viral medium. Cells proliferated for 6 hours in the viral medium. At 30 hours, the viral medium was removed and replaced by the complete medium. Cells were harvested 4 days post-transduction (PTD).

Western Blot: The western blot analysis was performed as previously described (Ueki et al., Dis Model Mech 11 (7), dmm033746, 2018). Briefly, cells were extracted on ice with lysis buffer (25 mM Tris-HCL, pH 7.5, 150 mM NaCl, 1 mM EDTA, 1% NP40, 5% glycerol) containing 1×HALT protease inhibitor cocktail, 1 mM PMSF, and 10 UM Leupeptine. A Bradford 1× protein-dye reagent (Bio-Rad #5000205) was used to determine the protein concentration. 25 μg of protein was separated by electrophoresis in an SDS-polyacrylamide gel, and the proteins were transferred onto a PVDF membrane. The membrane was blocked for 1 hour in 5% non-fat dry milk (NFDM) in 1× Tris-buffered saline containing 0.1% Tween-20. The membrane was incubated with primary antibodies overnight at 4° C. The primary antibodies to rabbit anti-Elp1 (1:1500) (Anaspec, #AS_54494)) and rabbit anti-GFP (1:2000) (Invitrogen, RRID: AB_221569) were diluted in 1×TBS containing 5% BSA and 0.1% Tween-20. Following primary incubation, membranes were probed with a secondary antibody (Horseradish peroxidase-conjugated goat anti-rabbit IgG (Jackson Laboratories, #111-035-144), 1:10,000 dilution, in 1×TBS containing 5% NFDM and 0.1% Tween-20. Quantitative analysis was performed using Image J (NIH, USA) software, and bands were normalized to total protein and the housekeeping gene Gapdh (1:10,000, Millipore, NP_002037).).

RT-qPCR: Real-time PCR (RT-qPCR) was used to quantify mRNA collected from snap-frozen retinal tissue. Total RNA was isolated and purified using the Direct-zol RNA mini-prep kit (Zymo Research, #R2050). The RNA concentration and purity was measured using BioTek Epoch 2 microplate spectrophotometer. 25 ng of purified RNA was reverse transcribed using SuperScript IV VILO mastermix (Invitrogen, #11756050). qPCR was performed with cDNA equivalent of 25 ng of transcribed RNA using SYBR Green master mix (Bio-Rad, #336501) using the 7500 Fast Real-Time PCR system (Fisher Scientific, Applied Biosystems, #4351106). The cycling condition was performed per the manufacturer's protocol (Bio-Rad). All primers (Table 1) were designed using NIH Primer-Blast tool. The relative expression analysis was performed using the delta delta CT method (ΔΔCT) and expressed as fold change normalized to the average of the two housekeeping genes.

TABLE 1

| Sequence Name | | Seq 5′ to 3′ | Accession No. | SEQ ID NO: |
|---|---|---|---|---|
| ELP1 | F | CTCTGCAGTCTCAGCACACA | NM_003640.5 | SEQ ID NO: 9 |
| ELP1 | R | CTGCTCCAGGATTGGCTCAA | | SEQ ID NO: 10 |
| Elp1 | F | GGTGACAGTCTTTCGGCAGA | NM_026079.3 | SEQ ID NO: 11 |
| Elp1 | R | GATCAGCAGCCGGTAGGTAC | | SEQ ID NO: 12 |
| Cckar | F | TGAACAAACGCTTTCGCCTG | NM_009827 | SEQ ID NO: 13 |
| Cckar | R | TGGCTGTAGGAATACCGGGA | | SEQ ID NO: 14 |
| Ptger4 | F | CACCACCTCGCTGAGAACTT | NM_008965 | SEQ ID NO: 15 |
| Ptger4 | R | TCCTTTAGAGGCAGGCTCCT | | SEQ ID NO: 16 |
| Hprt | F | TCAGTCAACGGGGGACATAAA | NM_013556 | SEQ ID NO: 17 |
| Hprt | R | GGGGCTGTACTGCTTAACCAG | | SEQ ID NO: 18 |
| Actb | F | AACCCTAAGGCCAACCGTGAA | NM_007393 | SEQ ID NO: 19 |
| Actb | R | TCACGCACGATTTCCCTCTCA | | SEQ ID NO: 20 |
| Bdnf | F | ACTGCAGTGGACATGTCTGG | NM_007540 | SEQ ID NO: 21 |
| Bdnf | R | AGTTGGCCTTTGGATACCGG | | SEQ ID NO: 22 |

RT-PCR analysis of ELP1 transcripts: Retinae were snap-frozen in a dry-ice bath with 95% EtOH upon collection. Tissues were homogenized in ice-cold TRI reagent (Molecular Research Center, Inc., Cincinnati, OH, USA), using a TissueLyser (Qiagen). Total RNA was extracted using the TRI reagent procedure provided by the manufacturer. RNA quality and yield was determined using a Nanodrop ND-1000 spectrophotometer. Reverse transcription was performed with 500 ng of total RNA, random primers, and Superscript™III reverse transcriptase (Invitrogen). PCR was performed for the splicing analysis with the 3 μl of cDNA in a total volume of 20 μl with GoTaq Polymerase 2× (Promega) and 32 amplification cycles. Human-specific ELP1 primers (forward, 5′-CCTGAGCAGCAATCATGTG-3′ (SEQ ID NO: 23); reverse, 5′ TACATGGTCTTCGTGACATC-3′ (SEQ ID NO: 24)) were used to amplify human ELP1 expressed from the transgene. The PCR products were separated on 1.5% agarose gels for 2.5 hours at 90V. The relative amounts of WT and mutant (A20) ELP1 spliced isoforms in a single PCR were determined using ImageJ and the integrated density value for each band as previously described (Hims et al., Genomics, 90 (3): 389-96, 2007; Shetty et al., Hum Mol Genet, 20 (21): 4093-101, 2011). The relative proportion of the WT isoform detected in a sample was calculated as a percentage.

Intravitreal injections: Three-week-old mice were anesthetized by isoflurane inhalation. Prior to anesthesia the pupil was dilated with one drop of phenylephrine (Phenylepherine Hydrochloride Ophthalmic Solution, USP 10%; National Drug Code, [NDC] 42702-103-05, Paragon, BioTeck, Inc) and one drop of tropicamide (Tropicamide Ophthalmic Solution, USP 1%, [NDC] 17478-102-12, Akorn Pharmaceuticals).

Two different intravitreal injection techniques (Manual Hamilton Syringe and WPI-UMP3 with Mirco2T Injector) were used to determine the optimal system for viral delivery. Each eye was treated as an independent experimental endpoint. All control-treated eyes were injected with an AAV2 preparation expressing eGFP at 1.6×10⁹ or 8.0×10⁸ viral genomes. Experimental-treated eyes were tested in a dose-dependent manner to optimize transduction efficiency and RGC rescue. Experimental-treated eyes receiving the AAV2-U1a-Elp1 were injected with 1.6×10⁹, 1.77×10⁹, 8.3×10⁸, or 8.6×10⁸ viral genomes of murine Elp1. Experimental-treated eyes receiving the AAV2-U1a-ELP1 were injected with either 5.4×10⁸, 2.7×10⁸, or 2.7×10⁷ vector genomes of human ELP1.

Manual Hamilton Syringe: Using forceps, the eyelids were pushed back to expose the cornea. With a 32-G, a small hole was made at the margin of the cornea and sclera. This was necessary because the A 34-G needle (#W1690678) had a blunt tip. The 5 μl calibrated Hamilton #65 needle was inserted at the margin of the sclera and cornea and into the vitreous cavity and 1 μl of the viral solution was injected slowly into the cavity. The needle was left in position for another 15 s and removed slowly.

WPI-UMP3 with Mirco2T Injector (#0916C): Using forceps, the eyelids were pushed back to expose the cornea. A 36-G beveled needle (NF36BV) attached to a 10 μl Hamilton NANOFIL syringe (WPI, lot #08C) was inserted at the margin of the sclera and cornea and into the vitreous cavity. Each eye was injected with 600 nL of AAV preparation bilaterally with a 200 nL/sec delivery rate. The needle was left in position for another 10 s to allow the pressure in the injection system to equilibrate with the pressure in the eye and removed slowly. After all surgeries, Puralube® Vet Ointment (Dechra Veterinary Products) was placed on the eye per IACUC guidelines. Mice were monitored and kept on a 37° C. heating pad until awake and recovered and then transferred to their home cage and monitored for 1 to 2 hours. Mice remained in their home cage with cage mates until analysis, 3 months after treatment.

Retinal histology following ocular gene therapy with AAV2-U1a-mElp1: Retina flat mount dissection and histology were performed as previously described (Cheng et al., Proceedings of the National Academy of Sciences, 117 (23): 13094-13104, 2020; Cheng et al., Hum Gene Ther, 32 (13-14): 649-666, 2021). In brief, Mice were euthanized with CO2 and the temporal surface of the eye was marked with a green tattoo dye (Ketchum Manufacturing) prior to enucleation. After the eyes were enucleated, a hole was made at the sclera-cornea margin and fixed overnight at 4° C. in 4% PFA. Before antibody staining, the neural retinal layer was detached from the retinal pigment epithelium (RPE)/choroid layer. Rabbit anti-Brn3a (1:300; Synaptic Systems, Cat #411 003) was used as the primary antibody. Cy3 conjugated anti-rabbit (1:500, donkey) secondary antibody was purchased from Jackson Immuno Research. All antibodies were diluted in 1×PBS with 0.3% Triton X-100 and 5% bovine serum albumin (BSA, Cell Signaling Technology).

Retinal histology following ocular gene therapy with AAV2-U1a-hELP1: Mice were euthanized as stated above. Eyes were fixed at room temperature (RT) for 1 hour in 4% PFA. Retinae were then removed, with the temporal region marked by a small cut, and incubated in 4% PFA for an additional 15 minutes at RT. Tissue was permeabilized in 1×PBS containing 0.5% Triton X-100 for 30 minutes at RT. Non-specific binding was blocked by incubation with 5% BSA containing 0.5% Triton X-100 for 3 hours at RT. All retinae were incubated for 2 nights at 4° C. in a primary antibody solution containing 2% BSA, 0.2% Triton X-100, an anti-Brn3 antibody (1:200, Santa Cruz, #sc-6026), and an anti-ELP1 (IKAP) antibody (2 μm/ml, Invitrogen, #PA5-111296, RRID AB_2856706). After overnight incubation at 4° C. with secondary antibodies (Donkey anti-Goat IgG (H+L) Cross-Adsorbed Secondary Antibody, Alexa Fluor™ 647 and Donkey anti-Rabbit lgG (H+L) Highly Cross-Adsorbed Secondary Antibody, Alexa Fluor™ 568 from Thermo Fisher Scientific, catalog #A-21447, RRID AB_2535864 and catalog #A10042, RRID AB_2534017), four identical cuts were made to flatten the tissue into four lobes, and the tissue was mounted with Prolong Gold (Invitrogen).

RGC quantification: Flat mounts were imaged and tiled using a Leica DM6 Thunder microscope at a 10× magnification. For the AAV2-HELP1 injected retinae, flat mount images were obtained with a Leica DMi8 THUNDER imaging system using the navigator spiral scan at a 10× magnification. IMARIS software (Oxford Instruments) was used to quantify the number of Brn3a+ cells. To identify Brn3a+ cells, the spots module was used. The detected diameter was set at 8 μm in the peripheral region determined by a 1 mm2 box placed at the edge of each quadrant in regions with the most cell death. If a specific region was damaged, the RGCs directly adjacent were counted. If any complication with the intravitreal injection, retinal harvest, or histology resulted in overall damaged retinal tissue, the sample was excluded and omitted from the analysis. The experimenters were blind to genotype and treatment group for all RGC quantification. The total peripheral cell count is the sum of the cells counted in the four peripheral regions. All the Brn3a+ RGCs were analyzed and counted across the entire retina but only report data from the peripheral retina because the Pax6-Cre is not expressed in the central retina. Therefore, the loss of Elp1 is restricted only to the periphery (Ueki et al., Dis Model Mech, 11 (7), 2018).

Statistics: All data are expressed as mean±SEM. Column data are plotted as a scattered dot-bar plot to show variability, and n shows the sample size for each data set. GraphPad Prism (version 9) was used to make all graphs. P<0.05 was considered to represent a significant difference. Following confirmation of normal distribution, data were analyzed using an unpaired two-tailed student's t-test and/or one-way ANOVA followed by Tukey's post hoc test for multiple comparisons.

For AAV2-hELP1 injection data, normality was visually checked, and a non-normal distribution was found; this was confirmed with a Shapiro test (p=7.89e-8). Due to non-normal distribution, a Kruskal Wallis non-parametric test (p=0.0005) was run. Followed by a post hoc Dunn test with Bonferroni correction, significance was found only between controls and Pax6cre;Elp1^{flox/flox} mice, regardless of treatment. Since the sample size was small with unequal variance and the relationship between the uninjected Pax6cre;Elp1^{flox/flox} and the Pax6cre;Elp1^{flox/flox} receiving the higher dose of 5.4×10⁸ vg/eye was most interesting, normality was tested for and found to have a non-normal distribution. Therefore, using a Mann-Whitney U test, the null hypothesis that the distributions are equal was rejected and it was concluded there is a difference between uninjected Pax6cre;Elp1^{flox/flox} and the Pax6cre;Elp1^{flox/flox} that received the highest vector dose (p=0.02). All statistics were performed using GraphPad Prism (version 9) and R version 4.2.1 (R Core Team, 2022).

### VIII. CLOSING PARAGRAPHS

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. By way of example, a material effect would cause a statistically significant reduction in expression of hELP1 from a viral vector, such as a rAAV2 expression vector.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8th Edition (Ed. J. Law & R. Rennie, Oxford University Press, 2020).

What is claimed is:

1. An isolated nucleic acid molecule comprising:
a polynucleotide comprising a nucleic acid sequence encoding a human ELP1 (hELP1) protein;
a promoter sequence arranged to promote expression of the human ELP1 protein in a mammalian cell; and
a post-transcriptional regulatory element.

2. The isolated nucleic acid molecule of claim 1, wherein the promoter sequence promotes expression of the ELP1 protein in a mammalian neuron.

3. The isolated nucleic acid molecule of claim 2, wherein the promoter sequence promotes expression of the ELP1 protein in a central nervous system (CNS) neuron.

4. The isolated nucleic acid molecule of claim 3, wherein the promoter sequence promotes expression of the ELP1 protein in a retinal neuron.

5. The isolated nucleic acid molecule of any one of claims 1-4, wherein the isolated nucleic acid molecule comprises a recombinant AAV2 vector, and the polynucleotide is flanked by AAV ITR sequences.

6. The isolated nucleic acid molecule of claim 1, wherein the encoded human ELP1 protein comprises an amino acid sequence at least 95% identical to the sequence of SEQ ID NO: 7.

7. The isolated nucleic acid molecule of claim 1, wherein the nucleotide sequence encoding the human ELP1 protein has a sequence at least 95% identical to the sequence of SEQ ID NO: 6.

8. The isolated nucleic acid molecule of claim 1, wherein the nucleotide sequence encoding the human ELP1 protein comprises the sequence of SEQ ID NO: 6.

9. The isolated nucleic acid molecule of claim 5, wherein the promoter sequence is no more than about 300 nucleotides long.

10. The isolated nucleic acid molecule of claim 1, wherein the promoter sequence is from a small nuclear RNA protein (SNRPA).

11. The isolated nucleic acid molecule of any of claims 1-10, wherein the promoter sequence is a small nuclear RNA U1 promoter.

12. The nucleic acid molecule of claim 11, wherein the promoter sequence is a murine or human Ula promoter.

13. The nucleic acid molecule of claim 11, wherein the promoter has a sequence comprising or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell.

14. The nucleic acid molecule of claim 1, wherein the polynucleotide molecule comprises:
a nucleotide sequence at least 95% identical to SEQ ID NO: 6 and encoding a functional human ELP1 protein; and
a Ula promoter sequence arranged to promote expression of the human ELP1 protein.

15. The nucleic acid molecule of claim 1, wherein the polynucleotide molecule comprises:
a nucleotide sequence identical to SEQ ID NO: 6;
a Ula promoter sequence having the sequence SEQ ID NO: 4 arranged to promote expression of the human ELP1 protein; or
both.

16. The nucleic acid molecule of claim 13, wherein the polynucleotide molecule has a sequence at least 95% identical to SEQ ID NO: 1.

17. The nucleic acid molecule of claim 15, wherein the polynucleotide molecule has a sequence identical to SEQ ID NO: 1.

18. A recombinant AAV (rAAV) virion, comprising:
an AAV2 capsid; and
the nucleic acid molecule of any one of claims 1-17.

19. A recombinant AAV (rAAV) virion comprising:
an AAV2 capsid; and
a recombinant polynucleotide comprising a nucleic acid sequence encoding human ELP1 operably linked to a promoter that promotes expression of the human ELP1 in retinal neurons, flanked by AAV ITR sequences.

20. A recombinant adeno-associated serotype 2 (rAAV2) virion comprising:
a rAAV2 vector polynucleotide; and, contained therein:
a human ELP1 gene nucleic acid sequence (hELP1);
a Ula promoter sequence arranged to promote expression of human ELP1 protein from the hELP1 gene nucleic acid; and
a post-transcriptional regulatory element.

21. The rAAV of claim 20, in which the polynucleotide comprises a nucleotide sequence functionally equivalent to the nucleotide sequence of SEQ ID NO: 1 (rAAV2.U1a.hELP1).

22. A pharmaceutical composition comprising the rAAV of any one of claims 18-21, formulated for administration to a mammalian subject.

23. The pharmaceutical composition of claim 22, formulated for ocular administration.

24. Use of the rAAV of any one of claims 18-21, or the composition of claim 22 or 23, to express hELP1 protein in a cell the genome of which contains a mutated hELP1 gene.

25. The use of claim 24, wherein the cell is a central nervous system cell or a peripheral nervous system cell of a mammal.

26. The use of claim 24, wherein the cell is a neuron.

27. A method of increasing ELP1 expression in retinal cells of a mammalian subject in need thereof, the method comprising:
administering to the mammalian subject a pharmaceutical formulation comprising an AAV2-hELP1 vector comprising:
a AAV2 capsid, and
a recombinant polynucleotide comprising: a nucleic acid sequence encoding human ELP1 (hELP1) operably linked to a promoter that promotes expression of the hELP1 in retinal neurons, flanked by AAV ITR sequences;
wherein the AAV2-HELP1 vector is delivered to at least one target site in the subject.

28. The method of claim 27, wherein one or more retinal cells of the mammalian subject express a mutant ELP1 protein.

29. The method of claim 27, wherein one or more retinal cells of the mammalian subject exhibit reduced expression of ELP1 as compared to a normal subject.

30. The method of claim 27, wherein one or more retinal cells of the mammalian subject express an ELP1 protein with reduced functionality as compared to a normal subject.

31. The method of claim 27, wherein administering the pharmaceutical formulation to the mammalian subject results in enhanced survival and/or functionality of retinal cells of the mammalian subject.

32. A method of increasing ELP1 expression in cells of a mammalian subject in need thereof, the method comprising:
providing a pharmaceutical formulation comprising an AAV2-hELP1 vector comprising:
a AAV2 capsid, and
a recombinant DNA insert comprising a nucleic acid sequence encoding human ELP1; and
delivering the pharmaceutical formulation to at least one target site in the central nervous system (CNS) or in the peripheral nervous system (PNS) of the subject.

33. The method of claim 32, wherein one or more retinal cells of the mammalian subject express a mutant ELP1 protein.

34. The method of claim 32, wherein one or more retinal cells of the mammalian subject exhibit reduced expression of ELP1 as compared to a normal subject.

35. The method of claim 32, wherein one or more retinal cells of the mammalian subject express an ELP1 protein with reduced functionality as compared to a normal subject.

36. The method of claim 32, wherein administering the pharmaceutical formulation to the mammalian subject results in enhanced survival and/or functionality of retinal cells of the mammalian subject.

37. The method of claim 27 or 32, wherein the target site comprises at least one eye of the subject.

38. The method of claim 37, wherein the pharmaceutical formulation is administered to the eye of the mammalian subject by an intravitreal, suprachoroidal, subretinal, or intraocular route, optionally by injection.

39. The method of claim 37, wherein the method comprises delivering to the subject a dose of:
$$1 \times {10}^{9} - 1 \times {10}^{12} viral genomes ( vg ) / eye ;$$
$$1 \times {10}^{10} - 1 \times {10}^{11} vg / eye ;$$
$$or$$
$$1 \times {10}^{9} - 1 \times {10}^{10} vg / eye .$$

40. The method of claim 27 or claim 32, wherein treating ELP1 deficiency in the mammalian subject reduces or prevents death of a neuron in the CNS or in the PNS of the mammalian subject.

41. The method of claim 27 or claim 32, wherein the mammalian subject is a human subject.

42. The method of claim 41, wherein the human subject is diagnosed with familial dysautonomia (FD).

43. The method of claim 42, wherein the human subject has a mutated ELP1 gene.

44. The method of claim 27 or claim 32, wherein the nucleic acid sequence encoding hELP1 is an hELP1 cDNA.

45. The method of claim 32, wherein the recombinant DNA insert comprises from 5′ to 3′:
a first inverted terminal repeat (ITR),
a small nuclear RNA U1a promoter,
the nucleic acid sequence encoding functional human ELP1,
a poly A signal sequence, and
a second ITR.

46. The method of claim 32, wherein the recombinant DNA insert comprises:
a Ula promoter comprising or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell, and
a nucleic acid sequence at least 95% identical to SEQ ID NO: 6.

47. The method of claim 27 or claim 32, wherein the AAV2-hELP1 vector comprises the nucleotide sequence of SEQ ID NO: 1.

48. The method of claim 27 or claim 32, wherein the pharmaceutical formulation is delivered by injection or infusion.

49. The method of claim 27 or 32, wherein the pharmaceutical formulation is delivered to eye of the subject by an intravitreal, suprachoroidal, subretinal, or intraocular route, optionally by injection.

50. A method of increasing ELP1 protein expression in a central nervous system (CNS) cell or a peripheral nervous system (PNS) cell of a mammalian subject, comprising administering to the CNS or the PNS of the mammalian subject a viral expression vector comprising:
a human ELP1-encoding nucleic acid sequence; and
a small nuclear RNA U1a promoter arranged to promote expression of the human ELP1 protein in a cell of the CNS or the PNS.

51. The method of claim 50, wherein one or more CNS cells or PNS cells of the mammalian subject express a mutant ELP1 protein.

52. The method of claim 50, wherein one or more CNS cells or PNS cells of the mammalian subject exhibit reduced expression of ELP1 as compared to a normal subject.

53. The method of claim 50, wherein one or more CNS cells or PNS cells of the mammalian subject express an ELP1 protein with reduced functionality as compared to a normal subject.

54. The method of claim 50, wherein administering the viral expression vector results in enhanced survival and/or functionality of retinal cells of the mammalian subject.

55. The method of claim 50, wherein the viral expression vector is an AAV2 viral expression vector.

56. The method of claim 55, wherein the human ELP1-encoding nucleic acid has a sequence at least 95% identical to the sequence of SEQ ID NO: 6.

57. The method of claim 56, wherein the human ELP1-encoding nucleic comprises the sequence of SEQ ID NO: 6.

58. The method of claim 55 or claim 56, wherein the encoded human ELP1 protein has a sequence at least 95% identical to the sequence of SEQ ID NO: 7.

59. The method of claim 58, wherein the encoded human ELP1 protein comprises the sequence of SEQ ID NO: 7.

60. The method of claim 50, wherein the promoter is a ubiquitous promoter or a retinal cell-specific promoter.

61. The method of claim 50, wherein the promoter is a murine Ula promoter or a human Ula promoter.

62. The method of claim 61, wherein the promoter has a sequence comprising or consisting of the sequence of SEQ ID NO: 4, or a sub-sequence from SEQ ID NO: 5 that exhibits promoter function in a mammalian cell, or a sequence within positions 1-206 of SEQ ID NO: 5 that exhibits promoter function in a mammalian cell.

63. The method of claim 50, wherein the viral vector comprises an adeno-associated virus serotype 2 (AAV2) or an adeno-associated virus serotype 9 (AAV9) vector.

64. The method of claim 63, wherein the AAV2 vector comprises AAV2-U1a-Elp1.


SEQUENCE LISTING (24 sequences)

SEQ ID NO: 1 - DNA, 7215 bp, Synthetic construct
  source 1..7215: mol_type = other DNA
ggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactaccggtgccaccatgcgaaatctgaaattatttcggaccctggagttcagggatattcaaggtccagggaatcctcagtgcttctctctccgaactgaacaggggacggtgctcattggttcagaacatggcctgatagaagtagaccctgtctcaagagaagtgaaaaatgaagtttctttggtggcagaaggctttctcccagaggatggaagtggccgcattgttggtgttcaggacttgctggatcaggagtctgtgtgtgtggccacagcctctggagacgtcatactctgcagtctcagcacacaacagctggagtgtgttgggagtgtagccagtggtatctctgttatgagttggagtcctgaccaagagctggtgcttcttgccacaggtcaacagaccctgattatgatgacaaaagattttgagccaatcctggagcagcagatccatcaggatgattttggtgaaagcaagtttatcactgttggatggggtaggaaggagacacagttccatggatcagaaggcagacaagcagcttttcagatgcaaatgcatgagtctgctttgccctgggatgaccatagaccacaagttacctggcggggggatggacagttttttgctgtgagtgttgtttgcccagaaacaggggctcggaaggtcagagtgtggaaccgagagtttgctttgcagtcaaccagtgagcctgtggcaggactgggaccagccctggcttggaaaccctcaggcagtttgattgcatctacacaagataaacccaaccagcaggatattgtgttttttgagaaaaatggactccttcatggacactttacacttcccttccttaaagatgaggttaaggtaaatgacttgctctggaatgcagattcctctgtgcttgcagtctggctggaagaccttcagagagaagaaagctccattccgaaaacctgtgttcagctctggactgttggaaactatcactggtatctcaagcaaagtttatccttcagcacctgtgggaagagcaagattgtgtctctgatgtgggaccctgtgaccccataccggctgcatgttctctgtcagggctggcattacctcgcctatgattggcactggacgactgaccggagcgtgggagataattcaagtgacttgtccaatgtggctgtcattgatggaaacagggtgttggtgacagtcttccggcagactgtggttccgcctcccatgtgcacctaccaactgctgttcccacaccctgtgaatcaagtcacattcttagcacaccctcaaaagagtaatgaccttgctgttctagatgccagtaaccagatttctgtttataaatgtggtgattgtccaagtgctgaccctacagtgaaactgggagctgtgggtggaagtggatttaaagtttgccttagaactcctcatttggaaaagagatacaaaatccagtttgagaataatgaagatcaagatgtaaacccgctgaaactaggccttctcacttggattgaagaagacgtcttcctggctgtaagccacagtgagttcagcccccggtctgtcattcaccatttgactgcagcttcttctgagatggatgaagagcatggacagctcaatgtcagttcatctgcagcggtggatggggtcataatcagtctatgttgcaattccaagaccaagtcagtagtattacagctggctgatggccagatatttaagtacctttgggagtcaccttctctggctattaaaccatggaagaactctggtggatttcctgttcggtttccttatccatgcacccagaccgaattggccatgattggagaagaggaatgtgtccttggtctgactgacaggtgtcgctttttcatcaatgacattgaggttgcgtcaaatatcacgtcatttgcagtatatgatgagtttttattgttgacaacccattcccatacctgccagtgtttttgcctgagggatgcttcatttaaaacattacaggccggcctgagcagcaatcatgtgtcccatggggaagttctgcggaaagtggagaggggttcacggattgtcactgttgtgccccaggacacaaagcttgtattacagatgccaaggggaaacttagaagttgttcatcatcgagccctggttttagctcagattcggaagtggttggacaaacttatgtttaaagaggcatttgaatgcatgagaaagctgagaatcaatctcaatctgatttatgatcataaccctaaggtgtttcttggaaatgtggaaaccttcattaaacagatagattctgtgaatcatattaacttgttttttacagaattgaaagaagaagatgtcacgaagaccatgtaccctgcaccagttaccagcagtgtctacctgtccagggatcctgacgggaataaaatagaccttgtctgcgatgctatgagagcagtcatggagagcataaatcctcataaatactgcctatccatacttacatctcatgtaaagaagacaaccccagaactggaaattgtactgcaaaaagtacacgagcttcaaggaaatgctccctctgatcctgatgctgtgagtgctgaagaggccttgaaatatttgctgcatctggtagatgttaatgaattatatgatcattctcttggcacctatgactttgatttggtcctcatggtagctgagaagtcacagaaggatcccaaagaatatcttccatttcttaatacacttaagaaaatggaaactaattatcagcggtttactatagacaaatacttgaaacgatatgaaaaagccattggccacctcagcaaatgtggacctgagtacttcccagaatgcttaaacttgataaaagataaaaacttgtataacgaagctctgaagttatattcaccaagctcacaacagtaccaggatatcagcattgcttatggggagcacctgatgcaggagcacatgtatgagccagcggggctcatgtttgcccgttgcggtgcccacgagaaagctctctcagcctttctcacatgtggcaactggaagcaagccctctgtgtggcagcccagcttaactttaccaaagaccagctggtgggcctcggcagaactctggcaggaaagctggttgagcagaggaagcacattgatgcggccatggttttggaagagtgtgcccaggattatgaagaagctgtgctcttgctgttagaaggagctgcctgggaagaagctttgaggctggtatacaaatataacagactggatattatagaaaccaacgtaaagccttccattttagaagcccagaaaaattatatggcatttctggactctcagacagccacattcagtcgccacaagaaacgtttattggtagttcgagagctcaaggagcaagcccagcaggcaggtctggatgatgaggtaccccacgggcaagagtcagacctcttctctgaaactagcagtgtcgtgagtggcagtgagatgagtggcaaatactcccatagtaactccaggatatcagcgagatcatccaagaatcgccgaaaagcggagcggaagaagcacagcctcaaagaaggcagtccgctggaggacctggccctcctggaggcactgagtgaagtggtgcagaacactgaaaacctgaaagatgaagtataccatattttaaaggtactctttctctttgagtttgatgaacaaggaagggaattacagaaggcctttgaagatacgctgcagttgatggaaaggtcacttccagaaatttggactcttacttaccagcagaattcagctaccccggttctaggtcccaattctactgcaaatagtatcatggcatcttatcagcaacagaagacttcggttcctgttcttgatgctgagctttttataccaccaaagatcaacagaagaacccagtggaagctgagcctgctagactgaaagcttaataaaagatctttattttcattagatctgtgtgttggttttttgtgtgc

SEQ ID NO: 2 - DNA, 141 bp, Synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 141 bp, Synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 4 - DNA, 251 bp, Mus musculus
  source 1..251: mol_type = genomic DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 5 - DNA, 1277 bp, Homo sapiens
  source 1..1277: mol_type = genomic DNA
ctctccgcacgcgggctggagaagcgggtcctacgcacgctttgttgtcgcgctttgcctccgtccttgcccctactcccgccttacctgacttccttttcggaggaagatccttgagcagccgacgttgggacaaaggatttggagaaacccagggctaaagtcacgtttttcctcctttaagacttacctcaacacttcactccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaaggcaccttcgtggagcgggaccgcaagcgggagaagaggaagcccaagagccaggagaccccggccaccaagaaggctgtgcaaggcgggggagccacccccgtggtgggggctgtccaggggcctgtcccgggcatgccgccgatgactcaggcgccccgcattatgcaccacatgccgggccagccgccctacatgccgccccctggtatgatccccccgccaggccttgcacctggccagatcccaccaggggccatgcccccgcagcagcttatgccaggacagatgccccctgcccagcctctttctgagaatccaccgaatcacatcttgttcctcaccaacctgccagaggagaccaacgagctcatgctgtccatgcttttcaatcagttccctggcttcaaggaggtccgtctggtacccgggcggcatgacatcgccttcgtggagtttgacaatgaggtacaggcaggggcagctcgcgatgccctgcagggctttaagatcacgcagaacaacgccatgaagatctcctttgccaagaagtagcaccttttccccccatgcctgccccttcccctgttctggggccacccctttcccccttggctcagccccctgaaggtaagtccccccttgggggccttcttggagccgtgtgtgagtgagtggtcgccacacagcattgtacccagagtctgtccccagacattgcacctggcgctgttaggccggaattaaagtggctttttgaggtttggtttttcacaa

SEQ ID NO: 6 - DNA, 3996 bp, Homo sapiens
  source 1..3996: mol_type = genomic DNA
atgcgaaatctgaaattatttcggaccctggagttcagggatattcaaggtccagggaatcctcagtgcttctctctccgaactgaacaggggacggtgctcattggttcagaacatggcctgatagaagtagaccctgtctcaagagaagtgaaaaatgaagtttctttggtggcagaaggctttctcccagaggatggaagtggccgcattgttggtgttcaggacttgctggatcaggagtctgtgtgtgtggccacagcctctggagacgtcatactctgcagtctcagcacacaacagctggagtgtgttgggagtgtagccagtggtatctctgttatgagttggagtcctgaccaagagctggtgcttcttgccacaggtcaacagaccctgattatgatgacaaaagattttgagccaatcctggagcagcagatccatcaggatgattttggtgaaagcaagtttatcactgttggatggggtaggaaggagacacagttccatggatcagaaggcagacaagcagcttttcagatgcaaatgcatgagtctgctttgccctgggatgaccatagaccacaagttacctggcggggggatggacagttttttgctgtgagtgttgtttgcccagaaacaggggctcggaaggtcagagtgtggaaccgagagtttgctttgcagtcaaccagtgagcctgtggcaggactgggaccagccctggcttggaaaccctcaggcagtttgattgcatctacacaagataaacccaaccagcaggatattgtgttttttgagaaaaatggactccttcatggacactttacacttcccttccttaaagatgaggttaaggtaaatgacttgctctggaatgcagattcctctgtgcttgcagtctggctggaagaccttcagagagaagaaagctccattccgaaaacctgtgttcagctctggactgttggaaactatcactggtatctcaagcaaagtttatccttcagcacctgtgggaagagcaagattgtgtctctgatgtgggaccctgtgaccccataccggctgcatgttctctgtcagggctggcattacctcgcctatgattggcactggacgactgaccggagcgtgggagataattcaagtgacttgtccaatgtggctgtcattgatggaaacagggtgttggtgacagtcttccggcagactgtggttccgcctcccatgtgcacctaccaactgctgttcccacaccctgtgaatcaagtcacattcttagcacaccctcaaaagagtaatgaccttgctgttctagatgccagtaaccagatttctgtttataaatgtggtgattgtccaagtgctgaccctacagtgaaactgggagctgtgggtggaagtggatttaaagtttgccttagaactcctcatttggaaaagagatacaaaatccagtttgagaataatgaagatcaagatgtaaacccgctgaaactaggccttctcacttggattgaagaagacgtcttcctggctgtaagccacagtgagttcagcccccggtctgtcattcaccatttgactgcagcttcttctgagatggatgaagagcatggacagctcaatgtcagttcatctgcagcggtggatggggtcataatcagtctatgttgcaattccaagaccaagtcagtagtattacagctggctgatggccagatatttaagtacctttgggagtcaccttctctggctattaaaccatggaagaactctggtggatttcctgttcggtttccttatccatgcacccagaccgaattggccatgattggagaagaggaatgtgtccttggtctgactgacaggtgtcgctttttcatcaatgacattgaggttgcgtcaaatatcacgtcatttgcagtatatgatgagtttttattgttgacaacccattcccatacctgccagtgtttttgcctgagggatgcttcatttaaaacattacaggccggcctgagcagcaatcatgtgtcccatggggaagttctgcggaaagtggagaggggttcacggattgtcactgttgtgccccaggacacaaagcttgtattacagatgccaaggggaaacttagaagttgttcatcatcgagccctggttttagctcagattcggaagtggttggacaaacttatgtttaaagaggcatttgaatgcatgagaaagctgagaatcaatctcaatctgatttatgatcataaccctaaggtgtttcttggaaatgtggaaaccttcattaaacagatagattctgtgaatcatattaacttgttttttacagaattgaaagaagaagatgtcacgaagaccatgtaccctgcaccagttaccagcagtgtctacctgtccagggatcctgacgggaataaaatagaccttgtctgcgatgctatgagagcagtcatggagagcataaatcctcataaatactgcctatccatacttacatctcatgtaaagaagacaaccccagaactggaaattgtactgcaaaaagtacacgagcttcaaggaaatgctccctctgatcctgatgctgtgagtgctgaagaggccttgaaatatttgctgcatctggtagatgttaatgaattatatgatcattctcttggcacctatgactttgatttggtcctcatggtagctgagaagtcacagaaggatcccaaagaatatcttccatttcttaatacacttaagaaaatggaaactaattatcagcggtttactatagacaaatacttgaaacgatatgaaaaagccattggccacctcagcaaatgtggacctgagtacttcccagaatgcttaaacttgataaaagataaaaacttgtataacgaagctctgaagttatattcaccaagctcacaacagtaccaggatatcagcattgcttatggggagcacctgatgcaggagcacatgtatgagccagcggggctcatgtttgcccgttgcggtgcccacgagaaagctctctcagcctttctcacatgtggcaactggaagcaagccctctgtgtggcagcccagcttaactttaccaaagaccagctggtgggcctcggcagaactctggcaggaaagctggttgagcagaggaagcacattgatgcggccatggttttggaagagtgtgcccaggattatgaagaagctgtgctcttgctgttagaaggagctgcctgggaagaagctttgaggctggtatacaaatataacagactggatattatagaaaccaacgtaaagccttccattttagaagcccagaaaaattatatggcatttctggactctcagacagccacattcagtcgccacaagaaacgtttattggtagttcgagagctcaaggagcaagcccagcaggcaggtctggatgatgaggtaccccacgggcaagagtcagacctcttctctgaaactagcagtgtcgtgagtggcagtgagatgagtggcaaatactcccatagtaactccaggatatcagcgagatcatccaagaatcgccgaaaagcggagcggaagaagcacagcctcaaagaaggcagtccgctggaggacctggccctcctggaggcactgagtgaagtggtgcagaacactgaaaacctgaaagatgaagtataccatattttaaaggtactctttctctttgagtttgatgaacaaggaagggaattacagaaggcctttgaagatacgctgcagttgatggaaaggtcacttccagaaatttggactcttacttaccagcagaattcagctaccccggttctaggtcccaattctactgcaaatagtatcatggcatcttatcagcaacagaagacttcggttcctgttcttgatgctgagctttttataccaccaaagatcaacagaagaacccagtggaagctgagcctgctagac

SEQ ID NO: 7 - protein, 1332 aa, Homo sapiens
MRNLKLFRTLEFRDIQGPGNPQCFSLRTEQGTVLIGSEHGLIEVDPVSREVKNEVSLVAEGFLPEDGSGRIVGVQDLLDQESVCVATASGDVILCSLSTQQLECVGSVASGISVMSWSPDQELVLLATGQQTLIMMTKDFEPILEQQIHQDDFGESKFITVGWGRKETQFHGSEGRQAAFQMQMHESALPWDDHRPQVTWRGDGQFFAVSVVCPETGARKVRVWNREFALQSTSEPVAGLGPALAWKPSGSLIASTQDKPNQQDIVFFEKNGLLHGHFTLPFLKDEVKVNDLLWNADSSVLAVWLEDLQREESSIPKTCVQLWTVGNYHWYLKQSLSFSTCGKSKIVSLMWDPVTPYRLHVLCQGWHYLAYDWHWTTDRSVGDNSSDLSNVAVIDGNRVLVTVFRQTVVPPPMCTYQLLFPHPVNQVTFLAHPQKSNDLAVLDASNQISVYKCGDCPSADPTVKLGAVGGSGFKVCLRTPHLEKRYKIQFENNEDQDVNPLKLGLLTWIEEDVFLAVSHSEFSPRSVIHHLTAASSEMDEEHGQLNVSSSAAVDGVIISLCCNSKTKSVVLQLADGQIFKYLWESPSLAIKPWKNSGGFPVRFPYPCTQTELAMIGEEECVLGLTDRCRFFINDIEVASNITSFAVYDEFLLLTTHSHTCQCFCLRDASFKTLQAGLSSNHVSHGEVLRKVERGSRIVTVVPQDTKLVLQMPRGNLEVVHHRALVLAQIRKWLDKLMFKEAFECMRKLRINLNLIYDHNPKVFLGNVETFIKQIDSVNHINLFFTELKEEDVTKTMYPAPVTSSVYLSRDPDGNKIDLVCDAMRAVMESINPHKYCLSILTSHVKKTTPELEIVLQKVHELQGNAPSDPDAVSAEEALKYLLHLVDVNELYDHSLGTYDFDLVLMVAEKSQKDPKEYLPFLNTLKKMETNYQRFTIDKYLKRYEKAIGHLSKCGPEYFPECLNLIKDKNLYNEALKLYSPSSQQYQDISIAYGEHLMQEHMYEPAGLMFARCGAHEKALSAFLTCGNWKQALCVAAQLNFTKDQLVGLGRTLAGKLVEQRKHIDAAMVLEECAQDYEEAVLLLLEGAAWEEALRLVYKYNRLDIIETNVKPSILEAQKNYMAFLDSQTATFSRHKKRLLVVRELKEQAQQAGLDDEVPHGQESDLFSETSSVVSGSEMSGKYSHSNSRISARSSKNRRKAERKKHSLKEGSPLEDLALLEALSEVVQNTENLKDEVYHILKVLFLFEFDEQGRELQKAFEDTLQLMERSLPEIWTLTYQQNSATPVLGPNSTANSIMASYQQQKTSVPVLDAELFIPPKINRRTQWKLSLLD

SEQ ID NO: 8 - DNA, 48 bp, Synthetic construct
  source 1..48: mol_type = other DNA
aataaaagatctttattttcattagatctgtgtgttggttttttgtgt

SEQ ID NO: 9 - DNA, 20 bp, Synthetic construct
  source 1..20: mol_type = other DNA
ctctgcagtctcagcacaca

SEQ ID NO: 10 - DNA, 20 bp, Synthetic construct
  source 1..20: mol_type = other DNA
ctgctccaggattggctcaa

SEQ ID NO: 11 - DNA, 20 bp, Synthetic construct
  source 1..20: mol_type = other DNA
ggtgacagtctttcggcaga

SEQ ID NO: 12 - DNA, 20 bp, Synthetic construct
  source 1..20: mol_type = other DNA
gatcagcagccggtaggtac

SEQ ID NO: 13 - DNA, 20 bp, Synthetic construct
  source 1..20: mol_type = other DNA
tgaacaaacgctttcgcctg

SEQ ID NO: 14 - DNA, 20 bp, Synthetic construct
  source 1..20: mol_type = other DNA
tggctgtaggaataccggga

SEQ ID NO: 15 - DNA, 20 bp, Synthetic construct
  source 1..20: mol_type = other DNA
caccacctcgctgagaactt

SEQ ID NO: 16 - DNA, 20 bp, Synthetic construct
  source 1..20: mol_type = other DNA
tcctttagaggcaggctcct

SEQ ID NO: 17 - DNA, 21 bp, Synthetic construct
  source 1..21: mol_type = other DNA
tcagtcaacgggggacataaa

SEQ ID NO: 18 - DNA, 21 bp, Synthetic construct
  source 1..21: mol_type = other DNA
ggggctgtactgcttaaccag

SEQ ID NO: 19 - DNA, 21 bp, Synthetic construct
  source 1..21: mol_type = other DNA
aaccctaaggccaaccgtgaa

SEQ ID NO: 20 - DNA, 21 bp, Synthetic construct
  source 1..21: mol_type = other DNA
tcacgcacgatttccctctca

SEQ ID NO: 21 - DNA, 20 bp, Synthetic construct
  source 1..20: mol_type = other DNA
actgcagtggacatgtctgg

SEQ ID NO: 22 - DNA, 20 bp, Synthetic construct
  source 1..20: mol_type = other DNA
agttggcctttggataccgg

SEQ ID NO: 23 - DNA, 19 bp, Synthetic construct
  source 1..19: mol_type = other DNA
cctgagcagcaatcatgtg

SEQ ID NO: 24 - DNA, 20 bp, Synthetic construct
  source 1..20: mol_type = other DNA
tacatggtcttcgtgacatc